(19) United States
(12) Patent Application Publication
(10) Pub. No.: US 2026/0317833 A1
(43) Pub. Date: Oct. 15, 2026

(54) PERSONALIZED SYNTHETIC BIOMARKER PANELS

(71) Applicant: GUARDANT HEALTH, INC., Palo Alto, CA (US)

(72) Inventors: Scott R. BORTNER, Palo Alto, CA (US); Alejandra RODRIGUEZ, Palo Alto, CA (US)

(21) Appl. No.: 19/641,979

(22) Filed: Apr. 8, 2026

Related U.S. Application Data
(60) Provisional application No. 63/786,007, filed on Apr. 9, 2025.

(51) Int. Cl. C12Q 1/6886; G01N 33/575

(52) U.S. Cl. CPC C12Q 1/6886; G01N 33/575; C12Q 2600/158

(57) ABSTRACT

Methods and systems for detecting disease using personalized synthetic biomarkers. Subject-specific molecular data, including one or more of genomic, epigenomic, transcriptomic, proteomic, or metabolomic data, are analyzed to identify disease-associated biological activities. Based on these activities, a set of synthetic biomarkers is designed for the subject, wherein each biomarker is configured to generate a detectable signal in response to a target biological process. The synthetic biomarkers are administered to the subject or contacted with a biological sample, and resulting signals are detected using analytical assays. Detection of the signals enables identification, monitoring, or assessment of disease presence, progression, or recurrence. In certain embodiments, the methods are applied to cancer, including minimal residual disease (MRD) detection, and may incorporate longitudinal monitoring and adaptive updating of biomarker panels based on changes in subject-specific molecular profiles.

### CROSS-REFERENCE TO RELATED APPLICATIONS

This application claims the benefit of priority to U.S. Provisional Patent Application No. 63/786,007, filed Apr. 9, 2025, which is incorporated by reference herein in its entirety.

### BACKGROUND

Early disease detection is critical since often treatment is most effective before clinical symptoms appear. However, one of the biggest barriers to early disease detection lies in the extremely low abundance of biomarkers present during initial stages.

This is particularly true in cancer, where early tumors shed far fewer detectable molecules into the bloodstream or other bodily fluids compared to advanced malignancies. Due to their limited release, rapid degradation, short circulation times, and massive dilution in the blood volume, these signals often fall below the detection limits of current diagnostic tools. This fundamental scarcity, more than any technical limitation, has made early disease detection a major challenge and underscores the need for new strategies.

Minimal residual disease (MRD) refers to the small number of cancer cells that remain in the body after treatment. Clinical evidence suggests that circulating tumor DNA (ctDNA) is a valuable biomarker for detecting MRD also known “measurable or molecular residual disease.” For example, clinical trials, such as the lung TRACERx study, have utilized ctDNA profiling following surgery to identify patients with early-stage NSCLC who are at high risk for disease recurrence, demonstrating the potential of ctDNA as an adjuvant biomarker (Abbosh et al., 2020). Additionally, in colorectal cancer, ctDNA analysis post-curative intent surgery has been shown to be a strong prognostic marker of recurrence-free survival, emphasizing its role as a significant and independent predictor of recurrence. Therefore, the use of ctDNA for MRD detection could enable oncologists to better personalize adjuvant therapies, potentially sparing patients from unnecessary toxic treatments.

Despite their success, sensitivity of current disease screening test can be hindered by low concentrations of circulating biomarkers, disease heterogeneity, and the challenge of distinguishing disease-specific signals from background noise. Therefore, there is a need to improve disease detection methods using sensitive diagnostic tests.

### SUMMARY

The present disclosure generally provides systems and methods to utilize synthetic biomarkers including protease-activated synthetic biomarkers, small-molecule probes, or genetic constructs that transform low tumor signals into detectable, disease-specific readouts.

To improve MRD detection, synthetic biomarkers may be determined based on tumor specific profiles including genetic, epigenetic, and/or histological profiles. Alternatively, or additionally, synthetic biomarkers can be determined based on the subject's genetic risk using, population specific single nucleotide polymorphisms (SNPs) and/or polygenic risk scores.

Likewise, to improve disease screening methods, synthetic biomarkers can be determined based on the subject's genetic risk using, population specific single nucleotide polymorphisms and/or polygenic risk scores. Alternatively, or additionally, SNPs can be annotated for functional consequences. For example, the SNP may alter gene expression by affecting transcription factor binding, RNA stability, splicing efficiency, or regulatory element activity, leading to changes in protein levels or function. Similarly, a SNP can influence DNA methylation by creating or disrupting CpG sites, altering methylation patterns, and subsequently affecting gene expression by modifying chromatin accessibility and transcription factor binding. Subject-specific variants for a subject including Clonal Hematopoiesis of Indeterminate Potential (CHIP) variants, ultra rare point mutations, and/or private epigenetic variants may also be used to personalize synthetic biomarkers.

Additionally, the methods provided in the present disclosure can apply to screening, diagnosing, or assessing risk for other diseases including autism spectrum disorder (ASD), Alzheimer's disease, and cardiovascular disease.

Moreover, the disclosure provides systems and methods to monitor disease in subjects using sequencing data from a variety of sample types. Leveraging data across sample types to determine synthetic biomarkers can provide valuable insights into the biochemical characteristics of a patient's tumor and improve MRD signal detection. Obtaining a personalized biochemical profile for an individual's cancer may help to identifying new druggable biomarkers, including epigenetics-modifying enzymes.

In certain aspects, the disclosure relates to methods of detecting cancer recurrence in a subject, comprising: (a) obtaining a tissue sample from a tumor in the subject to determine a tumor profile; (b) determining at least one synthetic biomarker for the subject based on the tumor profile; (c) obtaining a blood sample from the subject post medical treatment for the tumor, wherein prior to obtaining the blood sample, the subject was administered at least one of the synthetic biomarkers determined in (b); and (d) detecting the synthetic biomarker in the blood sample, wherein a positive result indicates cancer recurrence, thereby enabling cancer recurrence detection in the subject.

In some embodiments, the synthetic biomarker comprises a reporter attached to a molecule via a cleavable linker such that cleavage of the linker by a cancer specific agent in the subject releases the reporter from the molecule into the circulation and other biofluids in the subject.

In other embodiments, the detecting comprises employing an analytical assay. In some embodiments, the assay is selected from the group consisting of mass spectrometry, ELISA, PCR, and a colorimetric assay.

In additional embodiments, the tumor profile comprises a genetic, epigenetic, transcriptomic, proteomic, metabolomic, fragmentomic, and/or histological profile.

In some embodiments, the synthetic biomarker comprises a carrier particle having one or more cleavable peptide linkers, each linker attaching at least one reporter to the carrier particle such that cleavage of the linker by a tumor-associated protease releases the reporter from the carrier particle into the circulation and other biofluids in the subject.

In other embodiments, the carrier particle comprises iron oxide nanoparticles (IONPs), polyethylene glycol (PEG), or iron oxide nanoworms. In yet other embodiments, the reporter comprises at least one expression vector, mass-barcoded reporters, DNA barcodes, ligand-encoded reporters, volatile organic compounds (VOCs), or gold nanocluster.

In additional embodiments, the expression vector expresses at least one secreted reporter protein. In some embodiments, the secreted reporter protein comprises an alkaline phosphatase, gaussian luciferase (Glu), a fluorescent protein, and/or engineered enzyme. In other embodiments, expression of the secreted reporter protein is driven by a cancer inducible promoter. In yet other embodiments, the cancer inducible promoter comprises one or more functional elements inducible by known or unknown cancer specific molecules. In some embodiments, the known or unknown cancer specific molecule comprise a transcription factor. In other embodiments, the cancer inducible promoter is activated based on dysregulated gene expression patterns characteristic of cancer cells. In additional embodiments, the cancer inducible promoter comprises TERT, cyclooxygenase 2, surviving, or mucin 1 promoter.

In additional embodiments, the mass-barcoded reporters are chemically synthesized small molecules incorporating at least one stable isotope to generate distinct signatures. In some embodiments, the distinct signatures are detectable by mass spectrometry. In other embodiments, the stable isotope comprises carbon-13, nitrogen-15, oxygen-18, and/or sulfur-34.

In additional embodiments, the agent comprises an enzyme. In some embodiments, the enzyme comprises a protease, oxidases, transferases, or ligases.

In yet other embodiments, the medical treatment for the tumor comprises any one or a combination of: surgery, radiation therapy, chemotherapy, immunotherapy, targeted drug therapy, hormone therapy, stem cell transplant, car t-cell therapy, radiofrequency ablation, cryoablation, precision medicine, and/or proton therapy.

Furthermore, the method may include monitoring cancer recurrence in the subject over a plurality of time intervals. In some embodiments, the time intervals comprise six months, one year, two years, three years, four years, six years, and/or seven years. In other embodiments, the time intervals are determined based on the subject's age, sex, lifestyle, physical or biological phenotypes, and/or overall health. In yet other embodiments, the time intervals comprise standard of care intervals.

In additional aspects, the disclosure relates to methods of detecting cancer recurrence in a subject, comprising: (a) obtaining a tissue sample from a tumor in the subject to determine a tumor profile; (b) determining at least one synthetic biomarker for the subject based on the tumor profile, the biomarker comprising a reporter attached to a molecule via a cleavable linker such that in vivo cleavage of the linker by a cancer specific agent releases the reporter into the circulation and other biofluids; (c) obtaining a blood sample from the subject at a time post medical treatment for the tumor, wherein prior to obtaining the blood sample, the subject was administered at least one of the synthetic biomarkers determined in (b), thereby enabling in vivo cleavage of the linker by the cancer specific agent in the subject and release of the reporter from the molecule into the circulation and other biofluids in the subject; and (d) detecting the reporter in the blood sample obtained in (c), wherein a positive result indicates cancer recurrence, thereby detecting cancer recurrence in the subject.

In some embodiments, the agent comprises an enzyme. In other embodiments, the enzyme is selected from the group consisting of: a protease, oxidases, transferases, and ligases. In additional embodiments, the enzyme is expressed in the tumor microenvironment. In some embodiments, the agent is a membrane surface protein. In some embodiments, the surface protein is a receptor.

In additional embodiments, the synthetic biomarker is further determined based on the subject's disease risk.

In additional aspects, the disclosure relates to method of generating a post-treatment assay to detect cancer recurrence in a subject, comprising: (a) obtaining a tissue sample from a tumor in the subject to determine a tumor profile; and (b) determining a set of synthetic biomarkers based on the tumor profile thereby generating a post-treatment assay to detect cancer recurrence in the subject.

In yet other aspects, the disclosure relates to methods of generating a post-treatment assay to detect cancer recurrence in a subject, comprising: (a) obtaining a tissue sample from a tumor in the subject to determine a tumor profile; and (b) determining a set of synthetic biomarkers based on the tumor profile and/or the subject's disease risk, thereby generating a post-treatment assay to detect cancer recurrence in the subject.

In some embodiments, the synthetic biomarkers comprise standardized or off-the-shelf synthetic biomarkers. In other embodiments, the synthetic biomarkers comprise personalized biomarkers determined based on one or more properties of the tumor in the subject.

In yet other embodiments, the tumor profile comprises a genetic, epigenetic, transcriptomic, proteomic, metabolomic, fragmentomic, and/or histological profile.

In additional aspects, the disclosure relates to methods of monitoring disease state in a subject using a diagnostic test, wherein the subject was administered at least one personalized synthetic biomarker, wherein the synthetic biomarker was determined based on the subject's disease risk.

In other aspects, the disclosure relates to methods of screening disease in a subject, comprising: (a) obtaining a biological sample from the subject, wherein the subject was administered a personalized synthetic biomarker, the synthetic biomarker determined based on the subject's disease risk; (b) detecting in the biological sample the synthetic biomarker administered to the subject; and (c) determining disease status in the subject by comparing levels of the synthetic biomarker in the subject to reference normal levels thereby screening disease in the subject.

In some embodiments, the disease risk comprises genetic susceptibility to disease. In other embodiments, the disease risk is determined based on the subject's genetic or epigenetic composition. In yet other embodiments, the genetic composition is determined based on single nucleotide polymorphisms (SNPs). In some embodiments, the disease risk is determined based on polygenic risk scores.

In additional embodiments, the screening is performed at regular time intervals based at least on overall health, life expectancy, and/or prior screening history. In some embodiments, the screening comprises routine screening or preventive cancer screening.

In additional aspects, the disclosure relates to methods of screening disease in a subject, comprising: (a) obtaining a biological sample from the subject to determine polygenic risk scores for the subject; (b) determining at least one synthetic biomarker for the subject based on the polygenic risk scores determined in (a); (c) obtaining a second biological sample from the subject, wherein prior to obtaining the biological sample, the subject was administered at least one of the synthetic biomarkers determined in (b); and (d) detecting the synthetic biomarker in the second biological sample, wherein a positive result indicates the presence of disease in the subject, thereby enabling disease screening in the subject.

In some embodiments, the synthetic biomarker comprises a reporter attached to a molecule via a cleavable linker such that cleavage of the linker by a cancer specific agent in the subject releases the reporter from the molecule into the circulation and other biofluids in the subject.

In other embodiments, the detecting comprises employing an analytical assay. In yet other embodiments, the assay is selected from the group consisting of: mass spectrometry, ELISA, PCR, and a colorimetric assay.

In additional embodiments, the synthetic biomarker comprises a carrier particle having one or more cleavable peptide linkers, each linker attaching at least one reporter to the carrier particle such that cleavage of the linker by a disease-associated protease releases the reporter from the carrier particle into the circulation and other biofluids in the subject. In some embodiments, the carrier particle comprises iron oxide nanoparticles (IONPs), polyethylene glycol (PEG), or iron oxide nanoworms.

In some embodiments, the reporter comprises at least one expression vector, mass-barcoded reporters, DNA barcodes, ligand-encoded reporters, volatile organic compounds (VOCs), or gold nanocluster. In some embodiments, the expression vector expresses at least one secreted reporter protein. In additional embodiments, the secreted reporter protein comprises an alkaline phosphatase, gaussian luciferase (Glu), a fluorescent protein, and/or engineered enzyme.

In other embodiments, expression of the secreted reporter protein is driven by a cancer inducible promoter. In yet other embodiments, the cancer inducible promoter comprises one or more functional elements inducible by known or unknown cancer specific molecules. In some embodiments, the known or unknown cancer specific molecule comprise a transcription factor.

In other embodiments, the cancer inducible promoter is activated based on dysregulated gene expression patterns characteristic of disease. In yet other embodiments, the cancer inducible promoter comprises TERT, cyclooxygenase 2, surviving, or mucin 1 promoter.

In additional embodiments, the disease is selected from the group consisting of: cancer, cardiovascular disease, Alzheimer's disease, autism spectrum disorder, rheumatoid arthritis, and Crohn's disease.

In additional aspects, the disclosure relates to methods for screening disease in a subject, comprising: (a) obtaining a first biological sample from the subject to determine polygenic risk scores for the subject; (b) determining at least one synthetic biomarker for the subject based on the polygenic risk scores determined in (a), the biomarker comprising a reporter attached to a molecule via a cleavable linker such that in vivo cleavage of the linker by a disease specific agent releases the reporter into the circulation and other biofluids; (c) obtaining a second biological sample from the subject, wherein prior to obtaining the second biological sample, the subject was administered at least one of the synthetic biomarkers determined in (b) thereby enabling in vivo cleavage of the linker by the disease specific agent in the subject and release of the reporter from the molecule into the circulation and other biofluids in the subject; and (d) detecting the molecule in the biological sample obtained in (c), wherein a positive result indicates presence of disease in the subject, thereby screening disease in the subject.

In some aspects, the disclosure relates to a method of detecting a disease state in a subject, comprising: (a) obtaining subject-specific data comprising at least one of genetic, epigenetic, transcriptomic, proteomic, or clinical data; (b) determining, using the subject-specific data, a personalized synthetic biomarker configured to generate a detectable signal in response to a disease-associated biological activity; (c) exposing the subject or a biological sample from the subject to the personalized synthetic biomarker; and (d) detecting the detectable signal, wherein detection of the signal is indicative of the presence, absence, or progression of the disease state.

In some aspects, the disclosure provides a personalized synthetic biomarker composition comprising: (a) a carrier or scaffold; (b) at least one cleavable linker responsive to a disease-associated biological activity; and (c) at least one reporter configured to generate a detectable signal upon cleavage of the linker, wherein the composition is customized based on subject-specific molecular data. In some embodiments, the subject-specific molecular data comprises tumor-derived sequencing data. In some embodiments, the reporter comprises a DNA barcode, mass-barcoded molecule, or expressed protein. In some embodiment, the cleavable linker is responsive to a protease. In some embodiments, the carrier comprises a nanoparticle or polymer scaffold.

In some aspects, the present disclosure relates to a system for generating personalized synthetic biomarkers, comprising: (a) a data processing module configured to receive subject-specific molecular data; (b) a model configured to identify disease-associated features from the molecular data; and (c) a design module configured to generate a synthetic biomarker based on the identified features, wherein the synthetic biomarker is configured to produce a detectable signal in response to a disease-associated biological activity. In some embodiments, the model comprises a machine learning model. In some embodiments, the molecular data comprises multi-omic data from multiple sample types. In some embodiments, the system further comprises a detection module configured to analyze the detectable signal.

In some aspects, the present disclosure relates to a method of detecting a disease state in a subject, comprising: (a) obtaining a biological sample from the subject; (b) contacting the biological sample with a synthetic biomarker configured to respond to a disease-associated biological activity; and (c) detecting a signal generated by the synthetic biomarker, wherein the signal indicates the presence of the disease state.

In some aspects, the present disclosure provides a panel of synthetic biomarkers comprising a plurality of synthetic biomarkers, wherein each synthetic biomarker is configured to respond to a different disease-associated biological activity, and wherein the panel is personalized based on subject-specific molecular data. In some embodiments, the biomarkers correspond to tumor-specific mutations or epigenetic. In some embodiments, the panel is configured to generate a multiplexed detectable signal.

In some aspects, the present disclosure relates to a method of screening a subject for disease, comprising: (a) determining a disease risk profile for the subject based on genetic or epigenetic data; (b) selecting a synthetic biomarker based on the disease risk profile; and (c) detecting a signal generated by the synthetic biomarker; wherein the signal indicates the presence or likelihood of disease.

In some aspects, the disclosure relates to a method of detecting a disease state in a subject, comprising: (a) obtaining subject-specific molecular data comprising at least one of genomic, epigenomic, transcriptomic, proteomic, or fragmentomic data; (b) identifying one or more subject-specific disease-associated biological activities based on the molecular data; (c) generating a personalized set of synthetic biomarkers configured to respond to the identified biological activities; (d) contacting the subject or a biological sample from the subject with the personalized set of synthetic biomarkers; and (e) detecting one or more signals generated by the personalized set of synthetic biomarkers, wherein the detected signals are indicative of the disease state.

In some aspects, the present disclosure relates to a method of generating a personalized synthetic biomarker panel, comprising: (a) integrating a plurality of molecular datasets from a subject, the datasets comprising at least two of genetic, epigenetic, transcriptomic, proteomic, or metabolomic data; (b) identifying a set of disease-associated features based on the integrated datasets; and (c) selecting or designing a plurality of synthetic biomarkers configured to respond to the set of disease-associated features, wherein the plurality of synthetic biomarkers forms a personalized biomarker panel for the subject. In some embodiments, the integration comprises a machine learning model. In some embodiments, the datasets are derived from multiple sample types. In some embodiments, the disease-associated features comprise protease activity or transcription factor activity.

In some aspects, the present disclosure provides for a method of selecting synthetic biomarkers for disease detection, comprising: (a) determining a disease risk profile for a subject based on genetic or epigenetic data; (b) selecting a subset of synthetic biomarkers from a larger library based on the disease risk profile; and (c) detecting signals generated by the selected subset of synthetic biomarkers, wherein the signals are indicative of the presence or likelihood of disease.

In some aspects, the present disclosure provides for a method of monitoring disease in a subject, comprising: (a) obtaining first molecular data from the subject; (b) generating a first set of personalized synthetic biomarkers based on the first molecular data; (c) detecting signals from the first set of synthetic biomarkers; (d) obtaining second molecular data from the subject at a later time point; (e) updating the set of synthetic biomarkers based on the second molecular data; and (f) detecting signals from the updated set of synthetic biomarkers, wherein changes in the detected signals over time indicate disease progression or recurrence.

In some aspects, the present disclosure relates to a personalized synthetic biomarker panel comprising a plurality of synthetic biomarkers, each configured to respond to a different disease-associated biological activity, wherein the plurality of synthetic biomarkers is selected based on subject-specific molecular data to maximize detection sensitivity for the subject. In some embodiments, the biological activities comprise protease activity, transcription factor activity, or epigenetic regulation. In some embodiments, the panel is optimized using a computational model.

In some aspects, the disclosure relates to a system for generating personalized synthetic biomarkers, comprising: a data input module configured to receive subject-specific molecular data; a feature identification module configured to identify disease-associated biological activities; and a biomarker design module configured to generate a personalized synthetic biomarker panel based on the identified activities. In some embodiments, the disease-associated biological activities are selected based on one or more criteria, such as the type of somatic or epigenetic mutation, magnitude of dysregulation, specificity to disease tissue, known mutations associated with certain cancer types. These activities can be assigned a specific threshold and compared to a set threshold, ranking feature, and/or scoring function. In some embodiments, the disease-associated biological activities are updated based on longitudinal data from the subject.

In some aspects, the present disclosure relates to a method comprising: providing a library of synthetic biomarkers, each responsive to a different biological activity; selecting a subset of the synthetic biomarkers based on subject-specific molecular data; and using the selected subset to detect a disease state in the subject.

### DETAILED DESCRIPTION

Current disease screening tests are limited in achievable performance, limited patient adherence or adoption, and susceptibility to error. Synthetic biomarkers are an emerging class of engineered molecules designed for in vivo administration, to enhance disease-associated signals. Their tumor specific activation makes them highly sensitive with potentially greater diagnostic sensitivity than naturally shed endogenous biomarkers.

The present disclosure provides systems and methods to design and use synthetic biomarkers including engineered probes, vectors, or genetically recombined constructs.

Synthetic biomarkers may also comprise engineered nanosensors composed of protease-sensitive peptide substrates conjugated to carrier molecules such as polyethylene glycol (PEG) or other nanoparticles, and may be tagged with one or more mass-encoded or fluorescent reporters. These substrates may be selected based on their specific cleavage by tumor or disease-associated proteases, such as matrix metalloproteinases (MMPs) and cathepsins, which are typically overexpressed in the tumor microenvironment. Following administration, the synthetic biomarkers may circulate systemically, reaching both healthy and diseased tissues or fluids. Their personalized design ensures activation preferentially at tumor or disease sites, where overexpressed proteases cleave peptide substrates, releasing reporter fragments. These reporters may be designed to be small and hydrophilic, allowing them to quickly enter the bloodstream and other fluids. Once excreted, these reporters can be non-invasively collected from bodily fluids including blood, saliva, urine, sweat, cerebrospinal fluid, tears, semen, vaginal fluid, breast milk, amniotic fluid, synovial fluid, pleural fluid, peritoneal fluid, pericardial fluid, and/or lymph. Detection methods may include liquid chromatography, mass spectrometry (LC-MS/MS), proteomic assays-followed by sequencing or fluorescence-based detection. The concentration and/or identity of the reporters provide a multiplexed protease activity fingerprint that reflects MRD, tumor and/or disease presence and potentially its subtype. For example, concentration and identity may be indicative of Luminal A, Luminal B, HER2-enriched, Triple-negative, Basal-like, Claudin-low, Normal-like breast cancers.

A personalized panel of synthetic biomarkers avoids delivery of unnecessary biomarkers focusing on the subject's genetic susceptibility first. A variety of methods can be used to determined genetic susceptibility including polygenetic risk scores or sequencing data followed by analysis of individual Genome-wide association studies (GWAS) SNPs known to contribute to disease especially common genetic diseases. Common genetic diseases include cancer, type 2 diabetes, autoimmune disease, Crohn's disease cardiovascular disease, Alzheimer's, bipolar disorder, and schizophrenia.

In exemplary protocols, a personalized panel may be designed based on the subject's unique genetic, epigenetic, proteomic, and/or metabolomic composition. For example, a subject with genetic susceptibility to breast cancer, would benefit from a panel of synthetic biomarkers configured to activate in the breast cancer environment.

Additional analytical techniques to detect the released reporter may include mass spectrometry, ELISA, PCR, or colorimetric assays. The output can then be quantified and compared to reference patterns or thresholds, which can include readout from normal samples.

Screening test may use one or more synthetic biomarker to detect cancer in a variety of sample types including cell-free DNA from blood, stool, semen, saliva, and/or cerebral spinal fluid. Forms of synthetic biomarker administration may include intravenous injection, subcutaneous injection, oral administration, inhalation, local injection, and/or intravesical delivery.

Additionally, in certain aspects, the methods described herein are directed to targeted treatment selection for cancer positive patients to determine treatment options for these patients. Obtaining a molecular profile of the tumor using a variety of sample types may help detect cancer signal of origin, tissue of origin, cancer subtype, and/or cancer stage. Furthermore, the method can improve and/or accelerate the development of targeted treatments. In some embodiments, the disclosure provides systems and methods to develop biomarker tests leveraging epigenetic signatures in cancer positive patients.

In some embodiments, epigenetic data used to develop synthetic biomarkers from tumor specific profiles can comprise DNA epigenetic modifications, RNA epigenetic modifications, and/or proteomic epigenetic modifications. Epigenetic modifications can include methylation data, histone modification data, chromatin conformation capture data, nucleosome positioning, histone variants for example, replacement of the canonical histone H2A with the variant H2A.Z, RNA methylation, chromatin accessibility, DNA hydromethylation, DNA phosphorylation, acetylation, transcription factor binding sites, and/or chromatin looping or DNA-DNA interactions data. In some embodiments, of the disclosure the TFBS are directly assayed using methods such as ChIP-seq or CUT&Tag, in alternative embodiments the TFBS are inferred based on epigenetic data sets and/or machine learning algorithms. Epiproteomic posttranslational modifications can include involve histone acetylation, SUMOylation, phosphorylation, and ubiquitination. Additionally subject specific epigenetic profiles may be used to determine personalized biomarkers.

Additionally, the disclosure provides methods to monitor response to targeted treatment, diagnose, stage, and/or subtype a disease by monitoring changes in disease specific genetic and/or epigenetic biomarkers using ultra-low or low-depth sequencing. In some embodiments, sequencing depth can range between 0X-10×, 10×-100×, 100×-1000×, 1000×-10,000×, 10,000×-100,000×, 100,000×-1,000,000×. Furthermore, sequencing depths of at least 1000×can also be employed.

Methylation of CpG sites can vary across fragments, including fragments where 0-10% of the CpG sites are methylated, fragments where more than 10% and up to 20% of the CpG sites are methylated, fragments where more than 20% and up to 30% of the CpG sites are methylated, fragments where more than 30% and up to 40% of the CpG sites are methylated, fragments where more than 40% and up to 50% of the CpG sites are methylated, fragments where more than 50% and up to 60% of the CpG sites are methylated, fragments where more than 60% and up to 70% of the CpG sites are methylated, fragments where more than 70% and up to 80% of the CpG sites are methylated, fragments where more than 80% and up to 90% of the CpG sites are methylated, fragments where more than 90% of the CpG sites are methylated, and fragments where 100% of the CpG sites are methylated.

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### Definitions

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) into a fluid found in the organism, and may be obtained by obtaining a sample of the fluid without the need to perform an in vitro cell lysis step.

As used herein, a modification or other feature is present in “a greater proportion” in a first subsample or population of nucleic acid than in a second subsample or population when the fraction of nucleotides with the modification or other feature is higher in the first subsample or population than in the second population. For example, if in a first subsample, one tenth of the nucleotides are mC, and in a second subsample, one twentieth of the nucleotides are mC, then the first subsample comprises the cytosine modification of 5-methylation in a greater proportion than the second subsample.

As used herein “therapeutically targetable biomarkers” are biological molecules, such as proteins, genetic or epigenetic mutations, or other measurable molecular phenotypes that play a role in disease processes and can be directly targeted by specific treatments.

As used herein, “engineered feature(s)” refers to a feature created from raw data through transformation, combination, or extraction processes. These features are generated using domain knowledge to enhance the performance and predictive power of machine learning or artificial intelligence approaches. Engineered features can include interaction terms, polynomial features, log-transformed variables, binned categories, normalized or standardized values, text length, sentiment scores, frequency counts, ratio features, difference features, one-hot encoded variables, principal components, lag features, rolling window statistics, feature crossings, encoded categorical variables, and other domain-specific indicators. Additionally, engineered features can include aggregated or summary statistics such as the mean, median, or date-time components, including day, month, year, and hour.

As used herein, “lagged variables” are a features created by taking the value of a variable at a previous time point and including it as a feature in the model at the current time point to enhance model performance by incorporating temporal dependencies. Examples of lagged variables can include sequencing data from adjacent cycles or previous reads, lagged molecular data (e.g., protein levels, RNA counts) as features, expression values from an earlier time point, or any other molecular phenotype or genotype, clinical data, imaging, or histology data that can be obtained or captured from biological samples. Additionally, lagged variables can refer to data collected at earlier time points.

In practice, including information from previous molecular states or measurements as done using lagged variables, can help a model capture temporal dependencies, trends, and patterns that improve predictive accuracy and reliability.

As used herein, “machine learning model” (or “model”) refers to a collection of parameters and functions, where the parameters are trained on a set of training samples or individual data points or instances used to train a machine learning model. These samples are part of the dataset that provides the model with examples of input data along with the corresponding output (for supervised learning), or just input data (for unsupervised learning). The parameters and functions may be a collection of linear algebra operations, non-linear algebra operations, and tensor algebra operations. The parameters and functions may include statistical functions, tests, and probability models. The training samples can correspond to samples having measured properties of the sample (e.g., genomic, epigenomic, transcriptomic, metabolites etc. data and other subject data, such as histology, imaging data and/or electronic medical health records, or insurance claim data), as well as known patient/sample metadata including classifications or labels for example molecular phenotypes or specific cancer or disease therapies. Other phenotypes can include patient biomedical information including “cardiovascular phenotypes” or “cardiovascular risk factors” such as weight, height, Body Mass Index (BMI), and other physical characteristics. Yet other phenotypes can include cancer risk factors including smoking, excessive alcohol consumption, poor diet, physical inactivity, obesity, genetic predispositions, exposure to harmful chemicals and radiation, chronic inflammation, certain infections (such as human papillomavirus, hepatitis B and C), hormonal imbalances, and advanced age. The model can learn from the training samples in a training process that optimizes the parameters (and potentially the functions) to provide an optimal quality metric (e.g., accuracy) for classifying new samples. A variety of advanced statistical and computational methods that can be employed as training functions including Expectation Maximization (EM) to find maximum likelihood estimates of parameters in probabilistic models, especially for models with latent variables, Maximum Likelihood Estimation (MLE) to estimate the parameters of a statistical model. MLE methods select the set of parameters that maximize the likelihood function i.e., the parameters under which the observed data is most probable. Bayesian Parameter Estimation Methods which incorporate prior knowledge in addition to the data at hand through the use of probability distributions. These include Markov Chain Monte Carlo (MCMC), Gibbs Sampling, Hamiltonian Monte Carlo (HMC), and Variational Inference (VI), or Gradient-Based Methods including Stochastic Gradient Descent (SGD) and the Broyden-Fletcher-Goldfarb-Shanno (BFGS) algorithm.

As used herein, “Deep learning models” refers to a collection of “architectures” or “algorithms” useful in scenarios where machine learning approaches may fall short, for example due to the complexity of the data (e.g., high dimensional genomics, epigenomics datasets used alone or in one or more combinations). Additional high dimensional data can include medical images such as MRIs, or histology reports. Deep learning models, especially CNNs, can automatically extract relevant features without manual feature engineering.

Exemplary applications for deep learning models include complex pattern recognition tasks for example, recognizing specific functional elements in genomics data, functional elements can include transcription factor binding sites (TFBS) or chromatin interaction sites for example promoter-enhancer interactions. Additionally, recognition of disease specific features in histology images including patterns or specific features of PDL-1 expression.

As used herein, “without substantially altering base-pairing specificity” of a given nucleobase means that a majority of molecules comprising that nucleobase that can be sequenced do not have alterations of the base pairing specificity of the second nucleobase relative to its base pairing specificity as it was in the originally isolated sample. In some embodiments, 75%, 90%, 95%, or 99% of molecules comprising that nucleobase that can be sequenced do not have alterations of the base pairing specificity of the second nucleobase relative to its base pairing specificity as it was in the originally isolated sample.

As used herein, “base pairing specificity” refers to the standard DNA base (A, C, G, or T) for which a given base most preferentially pairs. Thus, for example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

“Capturing” one or more target nucleic acids refers to preferentially isolating or separating the one or more target nucleic acids from non-target nucleic acids.

A “captured set” of nucleic acids refers to nucleic acids that have undergone capture.

A “target-region set” or “set of target regions” refers to a plurality of genomic loci targeted for capture and/or targeted by a set of probes (e.g., through sequence complementarity).

“Corresponding to a target region set” means that a nucleic acid, such as cfDNA, originated from a locus in the target region set or specifically binds one or more probes for the target-region set.

“Specifically binds” in the context of a probe or other oligonucleotide and a target sequence means that under appropriate hybridization conditions, the oligonucleotide or probe hybridizes to its target sequence, or replicates thereof, to form a stable probe: target hybrid, while at the same time formation of stable probe: non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Sequence-variable target region set” refers to a set of target regions that may exhibit changes in sequence such as nucleotide substitutions (i.e., single nucleotide variations), insertions, deletions, or gene fusions or transpositions in neoplastic cells (e.g., tumor cells and cancer cells).

“Epigenetic target region set” refers to a set of target regions that may show sequence-independent changes in neoplastic cells (e.g., tumor cells and cancer cells) or that may show sequence-independent changes in cfDNA from subjects having cancer relative to cfDNA from healthy subjects. Examples of sequence-independent changes include, but not limited to, changes in methylation (increases or decreases), nucleosome distribution, CTCF binding, transcription start sites, and regulatory protein binding regions. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. In some embodiments, the epigenetic target region set includes one or more genomic regions, where the epigenetic state (e.g., methylation state) of cfDNA molecules in these regions is unchanged in cancer, but their presence/quantity in blood indicates increased, aberrant presentation of cfDNA from certain tissue (e.g. cancer origin) into circulation.

A nucleic acid is “produced by a tumor” or ctDNA or circulating tumor DNA, if it originated from a tumor cell. Tumor cells are neoplastic cells that originated from a tumor, regardless of whether they remain in the tumor or become separated from the tumor (as in the cases, e.g., of metastatic cancer cells and circulating tumor cells).

The term “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence). In some embodiments, DNA methylation refers to addition of a methyl group to adenine, such as in N⁶-methyladenine. In some embodiments, DNA methylation is 5-methylation (modification of the 5th carbon of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### Biochemical Assays

### Hi-C Technique to Capture Chromatin Conformation

Comprehensive genomic profiling has advance our understanding of the molecular mechanisms underlying complex diseases. Beyond comprehensive genomic profiling, the disclosure provides methods to identify molecular profiles including genetic, epigenetic, transcriptomic, epitranscriptomic, proteomic, fragmentomic, and/or chromatin interactions, to capture the biochemical changes that occur in cancer positive patients. These methods can be used to uncover cellular heterogeneity and detect cells with metastatic potential and those that have gained the ability to initial secondary tumors in distant tissues away from the primary tumor. Additionally integrating such data across multiple sample types, including blood, stool, semen, saliva, and/or breast milk can improve overall test performance. Moreover, the methods can provide informative biomarkers for therapy selection providing patients with personalized treatment options to eliminate residual cancer cells. Additionally, the present disclosure provides systems and methods to utilize these datasets in determining a tumor profile which can be used to inform design synthetic biomarkers for a subject. These datasets can also be used to determine personalized (i.e., subject-specific or tumor-specific) variants, molecular states, and/or epigenetic states in a subject prior to disease screening. Subject specific molecular profiles can help determine which diseases a subject should be screen for and can directly inform synthetic biomarker design.

Three-dimensional chromatin organization varies among different cell types and plays a crucial role in gene regulation by bringing distant functional elements into close spatial proximity. These functional elements contribute to maintaining homeostasis in health and play a pivotal role in disease regulation. The implementations of techniques, architectures, frameworks, systems, processes, and computer-readable instructions described herein are directed to the analysis of three-dimensional chromatin organization as captured by chromatin conformation capture (3C) techniques including, 4C (Circular Chromosome Conformation Capture), 5C (Chromosome Conformation Capture Carbon Copy), Hi-C, ChIA-PET (Chromatin Interaction Analysis by Paired-End Tag Sequencing), Capture-C, Capture Hi-C, HiChIP, Micro-C.

Following is an exemplary workflow that outlines the steps in preparing a Hi-C library, from cell culture to final library preparation and quality control.

In exemplary workflows for the Hi-C technique cells are cultured and chromatin is crosslinked e.g., fix cells in 1% formaldehyde, quench with glycine, harvest by centrifugation, and store at −80° C. for future use. Crosslinking conditions are typically standardized to ensure consistency across experiments. Cells are then lysed, and chromatin is digested. For example, cells can be lysed with a Dounce homogenizer in the presence of cold hypotonic buffer supplemented with protease inhibitors and IGEPAL CA-630. Lysates are wash and resuspended in restriction enzyme buffer, chromatin is then solubilized with SDS and incubated at 65° C., quench SDS with Triton X-100, and digested with a restriction enzyme (e.g., HindIII). Biotin marking of DNA ends and blunt end ligation: DNA ends are labeled with biotin-14-dCTP using the Klenow fragment of DNA polymerase I. Then, DNA fragments are ligated in a diluted condition at 16° C. to favor intra-molecular ligation. DNA is then purified with the following steps, degradation of proteins with Proteinase K, extraction with phenol: chloroform, DNA precipitation, resuspension, and RNase A treatment to yield high-quality DNA. Following DNA purification several quality control steps can be performed to ensure the library meets quality metrics. For example, by profiling fragment size distribution and quantify DNA using Agilent Bioanalyzer or Agilent TapeStation systems. Biotin removal from un-ligated ends: T4 DNA polymerase is used to remove biotin-labeled ends that have not been ligated. DNA is then precipitated and washed to prepare for downstream applications. DNA fragmentation and size fractionation: DNA is fractionated based on size using the Covaris 8700 and AMPure XP beads to achieve the desired size distribution for sequencing. End repair and “A” tailing: DNA molecules are repaired for asymmetric breaks and prepare for Illumina adapter ligation by filling in overhangs, and adenylating the 3′ ends. Streptavidin pull-down of biotinylated Hi-C ligation products: Mix biotinylated Hi-C ligation products with streptavidin beads, wash, and prepare for Illumina adapter ligation. Paired-end adapter ligation and library amplification: Illumina paired-end adapters are then ligated while the DNA is bound to streptavidin beads, libraries are PCR amplified with minimal cycles to avoid PCR artifacts. Final quality control and library quantification: Assess the quality of the final amplified library and quantify it, ideally using a Bioanalyzer, before sequencing.

Moreover, the chromatin conformation capture experiment (Hi-C, 3C, 4C, capture Hi-C etc.) might utilize chromatin fragmentation methods that do not depend on sequence specificity. Exemplary protocols include TopoLink™ (Catalog #: 21010) from Dovetail Genomics (part of Cantata Bio). In certain embodiments, a targeted approach might be more desirable, for example, when the research question is focused on promoter-enhancer interactions. Exemplary protocols include a capture Hi-C protocol using Dovetail Genomics Dovetail® Targeted Enrichment Panels (Catalog #25013). In some embodiments, experimental workflows can combine one or more experiments in a single workflow using a variety of methods. For example, the ChIP-seq and the Hi-C workflows can be combined in one workflow using Dovetail® HiChIP MNase Kit (Catalog #: 21007) see, for example, Yang, Jae-Hyun et al. “Loss of epigenetic information as a cause of mammalian aging.” Cell vol. 186,2 (2023): 305-326.e27. doi: 10.1016/j.cell.2022.12.027, which is incorporated herein by reference. This approach can investigate chromatin interactions mediated by specific proteins of interest. In additional embodiments, the methods disclosed herein can be used alone or in combination with Dovetail® Micro-C Kit (Catalog #21006) or similar methods, to generate uniform fragments that capture nucleosome positioning information, and maintain even coverage across the genome. This approaches obtain ultra-high-resolution topology mapping down to the mono-nucleosome level (150-200 bp conformation), see, for example, Bayanjargal, Ariunaa et al. “The DBD-α4 helix of EWS::FLI is required for GGAA microsatellite binding that underlies genome regulation in Ewing sarcoma.” bioRxiv: the preprint server for biology 2024.01.31.578127. 31 Jan. 2024, doi: 10.1101/2024.01.31.578127. Preprint which is incorporated herein by reference. Additionally, Hi-C experiments can improve and/or obtain haplotype phasing, genome assembly, and/or variant detection. Protocols and reagents to obtain such information include for example, Dovetail® Omni-C® Kit (Catalog #21006). See, for example, Milevskiy, Michael J G et al. “Three-dimensional genome architecture coordinates key regulators of lineage specification in mammary epithelial cells.” Cell genomics vol. 3,11 100424. 16 Oct. 2023, doi: 10.1016/j.xgen.2023.100424, which is incorporated herein by reference.

### Exemplary Workflows for the Capture Hi-C Technique

Cross-linking of Chromatin: Cells are treated with formaldehyde or a combination of cross-linkers to preserve physical interactions between chromosomal regions. Digestion of DNA: The cross-linked chromatin is then digested using a restriction enzyme. This step is crucial for creating ends that can be ligated later. Some protocols might use a combination of enzymes for more efficient digestion. Ligation under Dilute Conditions: The digested chromatin is diluted and ligated, allowing for the ligation of interacting DNA ends that are in close proximity due to chromatin folding. Purification and Shearing: Cross-links are reversed, and the DNA is purified. The DNA may then be sheared into smaller fragments to prepare for library preparation. Capture Step: Biotinylated probes, designed to hybridize to regions of interest, are used to selectively capture specific fragments from the ligated DNA pool. This step enhances the resolution and specificity of interactions being analyzed. Library Preparation: Captured DNA fragments are processed into a sequencing library, including end repair, A-tailing, adapter ligation, and enrichment of targeted fragments through biotin-streptavidin pull-down. Sequencing: The prepared library is sequenced using high-throughput sequencing technology. Capture-Hi-C data analysis involves processing sequencing data is to identify chromatin interactions, including mapping reads to a reference genome, filtering, and identifying significant interactions within the captured regions.

### Detailed Bioinformatic Pipeline for Hi-C Data

Various bioinformatic pipelines exists for the analysis of Hi-C datasets. Exemplary pipelines include, snHiC (Gregoricchio & Zwart, 2023). Briefly the analysis steps include (1) Generation of Contact Matrices: snHiC facilitates the creation of contact matrices at multiple resolutions in a single run, streamlining the initial analysis of Hi-C data. (2) Aggregation of Individual Samples: The pipeline allows for the aggregation of individual samples into user-specified groups, enabling comparative analyses across different conditions or time points. (3) Detection of Chromatin Features: It includes steps for the detection of domains, compartments, loops, and stripes, which are critical structural features of the genome organization revealed by Hi-C data. (4) Differential Analysis: snHiC supports differential compartment and chromatin interaction analyses, allowing users to identify changes in genome organization under different experimental conditions. The snHiC workflow can be automated using snakemake, which, makes it less prone to errors and more reproducible. To setup a yaml-formatted file is available to build a compatible conda environment, simplifying the setup and ensuring that users have all the necessary software and dependencies.

Similar analysis of Hi-C and Capture Hi-C data can be accomplished using publicly available algorithms including HiCUP: A pipeline for mapping and processing Hi-C and CHi-C data, removing artefacts and producing quality control reports. CHICAGO: Capture Hi-C Analysis of Genomic Organization. HiC-bench, a comprehensive and reproducible Hi-C data analysis platform. HiC-Pro optimized pipeline for processing Hi-C data from raw reads to normalized contact maps. ChiCMaxima, a pipeline for detection and visualization of chromatin looping in CHi-C, which also allows integrating information from biological replicates.

DNA methylation plays a crucial role in regulating gene expression and maintaining genome stability. Disruption of DNA methylation control mechanisms can lead to various diseases, including cancer. A body of evidence supports that cancer cells exhibit largely different DNA methylation patterns compared to normal cells. In general, cancer cells are characterized by genome-wide hypomethylation, as well as hypermethylation of CpG islands associated with tumor suppressor genes and developmental regulators. Hypermethylation in the promoter regions of tumor suppressor genes leads to their silencing, while hypomethylation in the promoter regions of oncogenes can activate them, both mechanisms play a significant role in the development of cancer.

### DNA Methylation Sequencing Workflows

Cellular adaptations that enable malignant cells to adapt include epigenetic changes including methylation. Various wet lab methods exist to obtain bisulfite converted DNA the following is an exemplary wet lab workflow. DNA Extraction and Quantification: Genomic DNA is extracted from the sample of interest, followed by quantification and quality assessment. Bisulfite Conversion: DNA is treated with sodium bisulfite, which converts unmethylated cytosines to uracil while leaving methylated cytosines unchanged. Library Preparation: The bisulfite-treated DNA is then used to prepare a sequencing library. Library preparation steps include fragmentation of DNA, end-repair, A-tailing, adapter ligation, and size selection. The library preparation method should be compatible with bisulfite-converted DNA, as the conversion process can degrade the DNA. PCR Amplification: The library is amplified by PCR to enrich for fragments that have adaptors ligated at both ends. Sequencing: The prepared library is sequenced using high-throughput sequencing technology. The choice of sequencing platform (e.g., Illumina, PacBio, or Oxford Nanopore) can vary based on the desired read length, throughput, and cost considerations. Data Analysis: Sequencing data is processed to identify and quantify methylation patterns across the genome. This involves aligning reads to a reference genome, identifying methylated cytosines, and performing downstream analyses such as differential methylation analysis between samples or groups. Moreover, integrating methylation across multiple sample types, including blood, stool, semen, saliva, and/or breast milk can improve overall test performance.

### DNA Methylation Data Analysis Tools

RnBeads is a software tool for large-scale analysis and interpretation of DNA methylation data Msuite: is an analysis toolkit for DNA methylation profiling, specifically optimized for emerging bisulfite-free methods. methylKit: An R package for the analysis of genome-wide DNA methylation profiles which also supports epigenome-wide association studies and biomarker discovery. BSmooth: Provides alignment, quality control, and analysis pipeline for whole-genome bisulfite sequencing. Similar open-source methods include MethLAB, MethCy and Methylation plotter. Additional methods include BEAT (BS-Seq Epimutation Analysis Toolkit), an R/Bioconductor package for quantitative analysis of DNA methylation from bisulfite sequencing data, utilizing a binomial mixture model. SINBAD, designed for pre-processing, quality assessment, and analysis of single-cell methylation data, starting from multiplexed sequencing reads. CpGtools, a Python package for analyzing DNA methylation data, offering a comprehensive suite for analyzing, annotating, QC, and visualizing the data and MethTools a toolbox for visualizing and analyzing DNA methylation data generated by the Bisulfite sequencing.

### Single Site Methylation Sequencing Workflows

Various single-site methylation (SSM) sequencing methods exist, these are broadly categorized into RRBS-based and WGBS-based approaches. RRBS-based methods focus on GC-rich regions, optimizing cost and efficiency for single-cell research. WGBS-based methods offer genome-wide coverage, with variations to enhance DNA preservation and reduce loss. Both methodologies include adaptations for single-cell analysis, integrating technologies like microfluidics and unique molecular identifiers to improve accuracy and minimize DNA loss. Workflows for single-site methylation sequencing has seen significant recent advances, with methodologies focusing on high-throughput, cost-efficiency, and enhanced sensitivity. A typical outline of the workflow based on the most recent research includes the following steps: (1) Sample Preparation and DNA Isolation: Collection of target samples, followed by DNA extraction and purification. This initial step is crucial for ensuring the quality of DNA for subsequent methylation analysis. Bisulfite Treatment (Optional for Some Methods): Traditional workflows often involve bisulfite conversion, where unmethylated cytosines are converted to uracil, while methylated cytosines remain unchanged. However, newer methods like MLAD-seq and EAC-seq offer bisulfite-free alternatives, providing single-base resolution and quantitative detection of 5mC without the DNA degradation associated with bisulfite treatment details of such protocols can be found in the literature for example Xiong et al., 2022 and Wang et al., 2022. Library Preparation and Sequencing: library preparation may involve enrichment of regions of interest or tagging DNA fragments with unique molecular identifiers (UMIs) before sequencing. Data Analysis and Interpretation: Post-sequencing, the data undergoes processing to identify methylated sites. Computational tools and algorithms, such as those incorporated in MethylScore, can accurately identify differentially methylated regions (DMRs) and predict phenotypes or disease states based on methylation profiles (Hüthe et al., 2022). Methods like EAC-seq be applied for direct and bisulfite-free detection of DNA methylation.

### Enzymes Used in Single Site Methylation Profiling

Bisulfite sequencing does not directly use enzymes for the conversion but relies on chemical treatment to differentiate between methylated and unmethylated cytosines. Methods that use enzymes include Ten-eleven translocation (TET)-assisted pyridine borane sequencing (TAPS): this method utilizes TET enzymes to oxidize 5mC to 5caC, which is then converted to thymine via pyridine borane reduction, allowing for sequencing without bisulfite conversion. Enzymatic methyl-seq (EM-seq) this method involves the use of enzymes for conversion, this approach is a less damaging alternative to bisulfite treatment for identifying methylated sites.

### Single Cell Methylation Profiling

Additionally, single cell methylation profiling techniques can be applied to one or more of the methods in the disclosure, including for example, MLAD-seq a technique for single-base resolution and quantitative detection of 5mC in DNA, EAC-seq utilizes engineered proteins for bisulfite-free, quantitative mapping of 5mC at single-base resolution, Digital-scRRBS a microfluidics-based platform for single-cell methylation sequencing, msRRBS a scalable single-cell reduced representation bisulfite sequencing technology that allows pooling of cell-specific barcoded DNA fragments before bisulfite conversion which improves efficiency and reduces cost.

### Methods to Error Correct DNA Methylation Data

Various methods exist to profile methylation status across the genome, including sodium bisulfite conversion and sequencing, differential enzymatic cleavage of DNA, and affinity-mediated capture of methylated DNA; however, these methods have limitations that may lead to misclassification, as the presence of unmethylated cytosines or DNA fragments may be erroneously recognized as methylated, and conversely, methylated cytosines may be detected as unmethylated. Additionally, the resolution of some techniques may not discern methylation variations in specific regions accurately. Therefore, there is a need for methods that can correct DNA methylation data, enhancing both specificity and sensitivity in analysis.

In additional aspects, the present disclosure provides methods to filter background noise in methylation data and to error correct DNA methylation data using chromatin interaction data to boost detection of disease associated signals. These methods work by leveraging the congruent methylation states of chromatin interaction partners Xi, Xii, Xiii . . . Xn, to identify and rectify inaccuracies in the methylation data. In essence, when interaction partners Xi and Xii are observed, they should consistently exhibit the same methylation status.

To address the noise in the methylation dataset, we construct a probabilistic model that captures the relationship between Xi and Xii and facilitates the removal of inaccuracies by emphasizing concordance and treating discordant instances as indicative of assay introduced errors not true biochemical states. An example of such model can comprise a linear model with an interaction or co-occurrence term. For example, given that Xi and Xii are interaction partners, methylation_status=β0+β1×Xi+β2×Xii+β3× (Xi×Xii)+ϵ. Where β3 represents the interaction or co-occurrence effect, indicating that the status of Xi depends on the status of Xii. In this model methylation status is the dependent variable and Xi status and Xii status are independent variables along with an interaction term. Individually, the terms in this linear model represent:

Methylation_status: Is the dependent variable predicted by the model.

β0: This is the intercept term representing the predicted value of methylation_status when both Xi and Xii are zero.

β1*Xi: This term represents the contribution of Xi to the predicted methylation_status. β1 is the coefficient associated the Xi determining the impact of a one-unit change on Xi on the predicted methylation_status.

β 2*Xii: Similarly, this term represents the contribution of Xii to the predicted methylation_status. β2 is the coefficient associated with Xii determining the impact of a one-unit change on Xii on the predicted methylation_status.

β 3*(Xi*Xii): is the interaction term, capturing the combined effect of Xi and Xii on the predicted methylation_status when they interact.

ϵ: Represents the error or residual in the model, capturing the difference between the predicted methylation_status and the actual observed values.

In essence, this model describes how Xi is influenced by its own value, the value of Xii, and the interaction between them. The coefficients β1, β2, β3 determine the strength and direction of these influences.

Alternatively, the relationship between the status of Xi, Xii, Xiii . . . Xn and their impact on the target variable methylation_status can be represented in a way that is not confined to a specific mathematical form. Exemplary models can include the function Methylation_status=f (Xi, Xii)-ϵ. Instead of using a predefined linear model, this is a more flexible approach using machine learning. By feeding a diverse set of data points into the model, we enable it to autonomously learn the relationship and dependencies between the features (Xi and Xii) and the target variable (methylation_status). In this scenario, we want the model to learn the congruence between the statuses of Xi and Xii and use this learned relationship to identify and correct discordant
methylation states between interaction partners in test data. In this context, training data can comprise a table listing all possible combinations of Xi and Xii such as Table 1.

TABLE 1
Possible combinations of Xi and Xii.

| Xi_status | Xii_Status | Correct_status |
|---|---|---|
| Methylated | Methylated | 1 |
| Methylated | Unmethylated | 0 |
| Unmethylated | Methylated | 0 |
| Unmethylated | Unmethylated | 1 |

Additionally, a training data can comprise chromatin interaction data comprising multiple interaction partners and the corrected status. Furthermore, the training data can comprise data with labeled examples of Xi and Xii and their corrected status, specifically tailored to the relationship between the variables.

Methylation_status=f(Xᵢ,Xᵢᵢ)—ϵ, where:

Methylation_status: represents the underlying relationship between Xi and Xii, it's the dependent variable to be predicted or output of the model.
f(Xᵢ, Xᵢᵢ): represents the function that the machine learning model learns.
ϵ: is the error term, accounting for any unexplained variability in the target variable that the model does not capture. The error term may also indicate that the observed value of the outcome variable may deviate from the predicted value due to random or unaccounted factors.

Such general function captures the relationship and interactions between Xi and Xii. Furthermore, this function can accommodate linear relationships, interactions, or more complex patterns based on the characteristics of the data. The target variable Methylation_status is determined by a relationship involving independent variables Xi and Xii. In this model, the actual form of the function f is not explicitly defined; it is determined by the learning algorithm based on the patterns in the training data. This allows the model to adapt to the specific characteristics of the data without imposing rigid assumptions about the underlying relationships.

In some cases, a concordance filter may be sufficient to filter out discordant Xi, Xii, Xiii . . . Xn interaction partners. A concordance filter can be a rule-based approach that leverages the knowledge that Xi and Xii should always have the same status (methylated or unmethylated) to filter out data points where their statuses between the pairs are discordant. A data structure comprising columns Xi status, Xii_status, and filtered where:

Xi_status: is a binary value (0 or 1) representing the status of Xi.

Xii_status: is a binary value (0 or 1) representing the status of Xii. filtered: is a Boolean flag (True/False) indicating whether the data point is filtered or not (this flag can be initially set to False for all).

Additionally, a filtering rule can be applied to the chromatin interaction data whereby we iterate through each data point and apply the following rule: If Xi_status is equal to Xii_status, set filtered to False (keep the data point). If Xi_status is not equal to Xii_status, set filtered to True (mark the data point as noise). This filtering rule approach assumes a perfect concordance between Xi and Xii, which might not always be true in chromatin interaction data and does not explicitly model the noise in the data collection process. However, assuming a perfect concordance between Xi and Xii, setting a filtering rule can improve the reliability of methylation assays based on the inherent relationship between one Xi, Xii or multiple interaction partners Xi, Xii, Xiii . . . Xn. As described earlier in this disclosure, to capture more complex relationships between Xi, Xii, Xiii . . . Xn, and other assay driven noise characteristics, machine learning models that learn the patterns in chromatin interaction data may be better suited. Other suitable options include Logistic Regression to predict the methylation status (methylated or unmethylated) of Xi based on both Xi status and Xii status. Alternately, a Decision Tree can be employed to learn a set of rules based on the methylation status or levels of methylation to classify data points as concordant or discordant. In additional embodiments, a Gaussian Mixture Models can be employed to model the joint distribution of Xi_status and Xii_status (methylation status or methylation level) to identify regions of high concordance and filter outliers.

### Methods to Integrate Sets of Molecular Data

The disclosure provides methods to identify molecular profiles including genetic, epigenetic, transcriptomic, epitranscriptomic, proteomic, fragmentomic, and/or chromatin interactions, to capture the biochemical changes that occur in disease including MRD. The molecular profile may then be used to develop synthetic biomarkers designed to amplify disease signals in a subject, including MRD that may remain after medical treatment or curative intent treatment. To better capture and integrate the multiple layers of biochemical information disease states, here, we describe a variety of artificial intelligence and machine learning approaches. Together these methods can provide the information necessary to develop personalized synthetic biomarkers and/or biomarkers for therapy selection providing cancer patients with personalized treatment options to eliminate malignant cells.

Multiple methods can be employed to integrate multiple layers of epigenome information such as methylation patterns, transcription factor binding sites (TFBS), and histone modifications described earlier in the disclosure to predict regional features like the presence of a TFBS or chromatin interaction sites (e.g., enhancer promoter interaction) at a specific genomic location. For example, a neural network approach can integrate multiple layers of epigenomic information to predict regional features, using deep learning to handle high-dimensional data. Such model can undergo continuous iteration and validation against known biological insights can refine the model specificity and sensitivity.

### Artificial Intelligence Analysis Tools

A myriad of biochemical changes occur in malignant cells, enabling them to survive both treatment and harsh conditions as they migrate and establish secondary tumors. Biochemical assays can profile these changes genome wide and interpreting these data requires advance artificial intelligence methods. Furthermore, integrating data from multiple sample types such as genetic data from blood and stool assays can improve overall assay performance. The present disclosure provides artificial intelligence methods to analyze a variety of datasets including genomic, proteomic, and clinical datasets. Additionally, the disclosure provides methods to integrate datasets from multiple sample types, clinical data, lifestyle data, and/or patient medical records.

### Neural Network Architecture: Multi-Modal Deep Learning Framework

Input Layer: the methods in the present disclosure provide datasets involving various types of epigenomic information from one or more sample types such as blood derived cell free DNA and or stool samples, hence the input layer is designed to handle multiple data modalities. We achieve this by using separate input channels or sub-networks for each data type (methylation, TFBS, and histone modifications). Each channel can preprocess its respective data type, normalizing and encoding it in a form suitable for deep learning, that is including several steps to convert raw data into a format that neural networks can effectively process and learn from. These steps include data normalization/standardization, encoding, reshaping, handling missing values, feature engineering/selection, and data augmentation. Feature extraction layers: For each data modality, a convolutional neural network (CNNs) or recurrent neural network (RNN) can be used to capture spatial dependencies and patterns within the genomic sequences. CNNs are particularly useful for identifying patterns in histone modifications and TFBS, while RNNs or transformer-based models can effectively process sequential data, capturing long-range dependencies in methylation patterns for example. Integration Layer: After feature extraction, the outputs of the separate channels are integrated. We achieve this through concatenation, followed by dense layers, or by using more sophisticated integration techniques like attention mechanisms, which allow the model to weigh the importance of information from different epigenomic layers dynamically. Prediction Layer: We then feed the integrated features into one or more dense layers with nonlinear activation functions to enable the prediction of regional features, for example the presence of a TFBS at a specific genomic location (this holds true for other feature like histone marks and interaction sites). The output layer is designed according to the specific prediction task, for example binary classification for predicting the presence/absence of a TFBS. Training: The model should be trained on labeled datasets where the ground truth (e.g., presence or absence of TFBS in specific regions or other functional element or genomic feature) is known. We use a cross-entropy loss function for classification tasks, and optimize the model using gradient descent algorithms like Adam or SGD. Regularization and Dropout: To prevent overfitting, given the complexity of epigenomic data and the deep architecture, we incorporate regularization techniques (L1/L2 regularization) and dropout layers, particularly after dense layers in the network. Evaluation and Fine-tuning: We evaluate the model's performance using standard metrics like accuracy, precision, recall, and F1 score. Depending on the results, fine-tune the model by adjusting the architecture, hyperparameters, or training procedure. In some embodiments we use techniques like cross-validation for a more robust evaluation. Similarly, for enhanced per Performance methods such as data augmentation techniques specific to genomic data to increase the diversity of the training set. Other techniques include multi-task learning when for example predicting multiple regional features simultaneously, under this framework the network is designed to make several predictions at once, sharing representations between tasks to improve learning efficiency and prediction accuracy. Lastly techniques such as transfer learning can be employed to leverage pre-trained models on related tasks to improve performance, this method is especially useful when labeled data are limited.

### Artificial Neural Network Training

Multi-Class Classification. An Artificial Neural Network (ANN) can be trained to predict the type of cancer including breast cancer, lung cancer, prostate cancer etc., based on a variety of datasets including gene expression levels, genomic and/or epigenetic markers, clinical, and/or life-style data. In exemplary embodiments, the data can be derived from multiple sample types to improve the overall test performance.

Genomic/Epigenomic Data Preprocessing. Genomic and epigenomic data, including, methylation, genomic and gene expression profiles, are first formatted to prepare for prepossessing. As an example, for RNA-seq data a numerical matrix where each row represents a sample (e.g., patient or tumor) and each column represents a feature (gene expression level for a specific gene). To feed into the neural network architecture, the data can then be preprocessed including normalization (scaling values to a range of [0,1] or standardizing to a mean of 0 and standard deviation of 1) and handling of missing values (imputation or removal). Optionally, feature selection can be performed to reduce dimensionality and focus on the most relevant genes. The preprocessed data can then be represented as input vectors, with each feature corresponding to a neuron in the input layer of the neural network, ready for downstream analysis.

Feeding Data into the Neural Network. Preprocessed genomic and epigenomic data can then be fed into the neural network, where each feature (e.g., gene expression value, methylation level) corresponds to a neuron in the input layer. Each sample is represented as a vector of feature values, which are processed by the input layer through weighted sums, bias addition, and activation functions such as ReLU, Sigmoid, Tanh, Leaky ReLU, etc., to introduce non-linearity and enable the network to learn complex patterns in the data.

Hidden Layers and Feature Transformation. After the input layer, the data is passed through one or more hidden layers, where each layer may apply a series of weighted sums, bias additions, and non-linear activation functions (e.g., ReLU) to transform the input features into higher-level representations. These hidden layers enabled the network to learn hierarchical patterns and abstract features from the input datasets, progressively refining its ability to distinguish between various classes or outcomes.

Output layer. The output layer of the ANN may comprise multiple neurons, including neurons for cancer type classification, metastasis prediction, therapy selection and/or outcome prediction. The network is trained to assign probabilities to each cancer type, and the one with the highest probability is the predicted outcome. Similarly for other outputs including cancer status, CSO, metastatic potential, and/or metastasis.

Hence the output layer may have one neuron for each cancer type (e.g., breast, colorectal, lung, etc.). These neurons may use a softmax activation function, which ensures that the outputs are probabilities that sum to 1. For example, if there are 5 cancer types, the output may look be: [0.1, 0.7, 0.05, 0.1, 0.05] i.e., probability of breast cancer: 10%, probability of colorectal cancer: 70%, probability of lung cancer: 5%, probability of brain cancer: 10%, probability of kidney cancer: 5%. Then, network is trained to assign higher probabilities to the correct cancer type based on the input data.

Output Layer Activation Functions. Depending on the specific task or output, each neuron in the output layer may employ a different activation function. For example, a neuron predicting cancer type might use a softmax activation for multiclass classification, while a neuron predicting survival risk could use a linear activation for regression. Similarly, if the neuron's output is a binary classification such as cancer status it may use a sigmoid activation function.

### Classifier Training and Pathway Enrichment Analysis

Cancer is a heterogeneous disease with malignant cells that exhibit distinct molecular profiles. Their continuous evolution allows these cells to survive treatment and gain the ability to initiate secondary tumors. Identifying distinct biochemical changes and/or disrupted biological pathways in cancer cells using multiple sample types such as blood and stool can help early disease detection, identification of cancer subtypes, accurately stage the disease, and determine the most appropriate treatment options. Additionally, these biochemical changes may also serve therapeutic targets to guide personalized treatment. In some embodiments of the disclosure sequencing data from a plurality of molecules including DNA, RNA, and their corresponding epi modifications i.e., epitranscriptomic biochemical modifications and epigenetic biochemical modifications may be used to identify disrupted pathways in malignant cancer.

In certain exemplary analysis pipelines, RNA-seq data is obtained from subjects undergoing or who have previously undergone curative intent treatment. Raw sequencing reads may undergo quality control using FastQC to assess sequencing quality, followed by adapter trimming with TrimGalore. Sequencing reads are then aligned to a suitable human genome reference for example hg38, using STAR, to generate BAM alignment. Alignment files may also undergo preprocessing prior to downstream analyses.

A variety of opensource software may be used to obtain gene expression levels from the samples including for example featureCounts. featureCounts (Subread package), quantifies the number of reads that align to specific genomic features to generate a matrix of transcript abundances or TPM values. In alternative pipelines, expression matrix may also be generated using HTSeq-count, RSEM, Salmon, Kallisto, StringTie, or Cufflinks.

Known biological pathways from public databases including KEGG and MSigDB may be used to calculate pathway activity scores from the expression matrix data. In certain embodiments the analysis may employ Pathway Activity Autoencoders (PAAE) to assign scores for 186 KEGG pathways thereby summarizing gene expression data into interpretable pathway-level activity or pathway activity scores.

Using the pathway activity scores as input data, a Random Forest classifier can be trained to predict cancer status for a test sample. In alternative embodiments activity scores and additional genomic data can be used as inputs into the classifier. Then the model may use the top pathways with the highest mutual information as features to achieve an acceptable Area Under the Receiver Operating Curve (AUC) on an independent validation dataset. Acceptable AUC will vary and can range from 0.50-0.99 or a maximum AUC of 1.0 can be obtained.

Additional predictive features may be used as inputs into the classifier including multi-omic datasets and clinical data. To achieve translational success, clinical trials should be conducted to validate findings. To improve performance and/or to integrate additional genomic and/or clinical features a Fully Connected Neural Networks (FCNN), Convolutional Neural Networks (CNN), Recurrent Neural Networks (RNN), Long Short-Term Memory Networks (LSTM), Autoencoders (AE), Variational Autoencoders (VAE), Graph Neural Networks (GNN), Transformers, Gradient Boosted Decision Trees with Neural Networks, or Hybrid models integrating neural networks with statistical techniques may also be used.

In some aspects, in a liquid biopsy assay, samples comprising methylation sequencing data from cancer positive and cancer negative patients may also be used alone or in combination with the pathway activity scores obtained from the RNA-seq data, to train the cancer classification model, and/or to determine therapeutic options for subjects who are undergoing or who have undergone curative intent treatment. Briefly, individual panel regions predictive of cancer status can be annotated for nearest gene, the list of genes can then be used to perform gene set enrichment analysis using the gseapy package (enrichr function) in python (or similar gene set enrichment analysis software) to identify enriched pathways in cancer patients who have undergone curative intent treatment and determining based on the enrichmed pathway information an cancer or disease status, risk of disease recurrence, and/or personalized treatment options. In certain embodiments of the disclosure, the inputs to the classifier are pathway activity scores determined from RNA-seq data and methylation sequencing data from patients who have undergone curative intent treatment.

Additional modules in the enrichr function can be used to identify relevant hub genes, TFBS, miRNAs in cancer or disease samples.

Curative intent treatment can include surgery, chemotherapy, radiation, targeted therapy, immunotherapy, hormone therapy, stem cell transplant, photodynamic therapy, cryoablation, and hyperthermia therapy used alone or in combination with each other.

### RNA Extraction and Isolation

In some embodiments of the disclosed methods, the population of target nucleic acids comprises RNA and the method further comprises a cDNA synthesis step. RNA for use in the methods disclosed herein may be isolated from a blood sample or a sample comprising cells (such as a sample that includes immune and/or cancer-derived cells (e.g., a blood sample such as a whole blood sample, a buffy coat sample, a leukapheresis sample, or a peripheral blood PBMC sample)). General methods for RNA extraction and isolation (such as mRNA extraction and isolation) are known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56: A67 (1987), and De Andrés et al., BioTechniques 18:42044 (1995). In particular, RNA isolation can be performed using a purification kit, buffer set, and protease(s) from commercial manufacturers, such as PreAnalytix GmbH or Qiagen, according to the manufacturer's instructions. For example, RNA can be extracted from whole blood samples using the PAXgene® Blood RNA Kit (PreAnalytix GmbH). Other commercially available RNA isolation kits include MasterPure Complete DNA and RNA Purification Kit (EPICENTRE, Madison, WI), and Paraffin Block RNA Isolation Kit (Ambion, Inc.). Total RNA from tissue samples can be isolated using RNA Stat-60 (Tel-Test). RNA prepared from tumor tissue can be isolated, for example, by cesium chloride density gradient centrifugation.

cDNA Library Preparation

Following RNA extraction from a sample (such as a blood sample), a cDNA library is typically prepared in preparation for sequencing, e.g., as in RNA-Seq. In some embodiments, the cDNAs in a library, such as an RNA-Seq library, can comprise a cDNA insert flanked by adapter sequences, such as adapter sequences used for amplification and sequencing on a particular platform. Exemplary cDNA library preparation methods are discussed below; however, cDNA library preparation methods can vary depending on the RNA species under investigation, which can differ in size, sequence, structural features and abundance. One of ordinary skill in the art will be able to select cDNA library preparation methods suitable for cDNA library preparation using an RNA species of interest.

rRNA and/or Globin mRNA Depletion; Poly(A) Selection

Ribosomal RNAs (rRNAs) are the most abundant RNA species in most cells. Globin mRNA is also abundant in certain cell types found in the blood. Thus, some embodiments of the present disclosure comprise a step of ribosomal RNA (rRNA) depletion and/or a step of globin mRNA depletion. Such steps can be performed, e.g., following RNA extraction from a sample, and prior to a step of RNA fragmentation or cDNA fragmentation, prior to a step preparing cDNA from the RNA, prior to a step of ligating adapters to the cDNA, and prior to a sequencing step. In some embodiments, the methods include a step of rRNA depletion. In other embodiments, the methods include a step of globin mRNA depletion. In yet other embodiments, the methods disclosed herein include both a step of rRNA depletion and a step of globin mRNA depletion.

Any suitable rRNA depletion and/or globin mRNA depletion methods are of use in the present disclosure. One approach is to eliminate rRNAs uses sequence-specific probes that can hybridize to rRNAs (Hrdlickova et al., Wiley Interdiscip Rev RNA. 2017; 8 (1): 10.1002/wrna.1364). Unwanted rRNAs or their cDNAs are hybridized with biotinylated DNA or locked nucleic acid (LNA) probes, followed by depletion with streptavidin beads. Alternatively, rRNAs can be targeted by anti-sense DNA oligos and digested by RNase H, a method also known as probe-directed degradation (PDD). Another approach for rRNA reduction uses specific, not-so-random (NSR) primers that bind to the RNA molecules of interest during reverse transcription, thus avoiding reverse transcription of the rRNAs. For example, a method known as Ovation RNA-Seq (NuGen) uses hexamer or heptamer primers whose sequences are not present in rRNAs. In addition to sequence-based approaches, some methods take advantage of certain features of rRNAs for their elimination. The COT-hybridization method is based on heat denaturation, re-annealing, and selective degradation by a duplex-specific nuclease (DSN). Double-stranded cDNAs from abundant sequences are preferentially degraded because of their more rapid annealing kinetics compared to less abundant ones. Selective degradation has also been achieved using the enzyme terminator 5′-phosphate-dependent exonuclease (TEX), which recognizes RNA molecules with 5′-monophosphate, as with rRNAs and tRNAs. Further, commercial kits are available for rRNA and globin mRNA depletion, including, e.g., the Watchmaker Genomics RNA Library Prep Kit with Polaris Depletion.

Other embodiments of the present disclosure comprise a step of poly(A) selection. Such a step can be performed, e.g., following RNA extraction from a sample, and prior to a step of RNA fragmentation or cDNA fragmentation, prior to a step preparing cDNA from the RNA, prior to a step of ligating adapters to the cDNA, and prior to a sequencing step. In eukaryotic organisms, most protein coding RNAs (mRNAs) and many long noncoding RNAs (lncRNAs) (>200 nt) comprise a poly(A) tail (“polyadenylated RNAs”). The poly(A) tail may be used to enrich for polyadenylated RNAs from total cellular RNA, in which polyadenylated RNAs may account for approximately 1-5% of total cellular RNA (Hrdlickova et al., Wiley Interdiscip Rev RNA. 2017; 8 (1): 10.1002/wrna. 1364). Exemplary poly(A) selection methods include, but are not limited to, use of magnetic or cellulose beads coated with oligo-dT molecules. Alternatively, polyadenylated RNAs can be selected using oligo-dT priming for reverse transcription (RT). Poly(A) selection may be combined with globin mRNA depletion.

### Fragmentation

In some embodiments, methods disclosed herein comprise fragmenting RNA isolated from a sample (such as RNA isolated from a sample comprising cells, such as a whole blood sample, a buffy coat sample, a leukapheresis sample, or a PBMC sample), such as following poly(A) selection or rRNA and/or globin mRNA depletion. RNA fragmentation methods can include physical fragmentation, chemical fragmentation, and/or enzymatic fragmentation. Physical fragmentation methods include, but are not limited to, acoustic or hydrodynamic shearing (such as sonication or point-sink shearing), needle shearing, and nebulization. Enzymatic fragmentation methods can include use of a ribonuclease (such as RNase III). RNA may also be fragmented using chemical shearing methods. Chemical fragmentation methods can include, but are not limited to, heat treatment of RNA in the presence of a divalent metal cation (such as magnesium or zinc). In some embodiments, the fragmenting provides RNA (such as mRNA) fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

Alternatively, non-fragmented RNAs can be reverse transcribed, and the resultant cDNA can be fragmented. cDNA fragmentation methods can include physical fragmentation, chemical fragmentation, and/or enzymatic fragmentation. Physical fragmentation methods include, but are not limited to, acoustic or hydrodynamic shearing (such as sonication or point-sink shearing), needle shearing, and nebulization. Enzymatic fragmentation methods can include use of a restriction endonuclease (such as a 4-cutter or 5-cutter restriction endonuclease, e.g., Alul, DpnI, Eco47I, HaeIII, HpaII, Mbo I, Msel, MspI, PspGI, RsaI, Sse9I, or TaqI), a non-specific nuclease (e.g., micrococcal nuclease), or a transposase (for example, when insertion of an adapter into a fragmented double-stranded cDNA molecule is desired). cDNA may also be fragmented using chemical shearing methods. Chemical fragmentation methods can include, but are not limited to, heat digestion of cDNA in the presence of a divalent metal cation (such as magnesium or zinc). In some embodiments, the fragmenting provides cDNA fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

cDNA Preparation

Some embodiments of the disclosed methods comprise preparing cDNA from RNA (such as RNA extracted from a blood sample), such as by reverse transcription of the RNA template into cDNA. Reverse transcription is generally followed by exponential amplification of the cDNA, e.g., in a PCR reaction. Two commonly used reverse transcriptases are avian myeloblastosis vims reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse transcribed using a Gene Amp RNA PCR kit (Perkin Elmer, Calif., USA), following the manufacturer's instructions. The derived cDNA can then be used as a template in the subsequent amplification (e.g., PCR) reaction. In some embodiments, RNA is converted to cDNA using random priming, followed by second strand synthesis, end repair, and optional A-tailing. Adapters comprising barcodes can then be ligated to the cDNA, which is then amplified.

Amplification is typically primed by primers that anneal or bind to primer binding sites in adapters flanking a cDNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence-based replication.

Although a PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

The primers used for the amplification are selected so as to amplify a unique segment of the gene of interest, such as RNA (such as mRNA) encoding a gene of a target gene set described herein. In some embodiments, expression of other genes is also detected, such as other known disease markers (such as known cancer markers) or housekeeping genes. Primers that can be used to amplify disease-related molecules are commercially available or can be designed and synthesized. In some examples, the primers specifically hybridize to a promoter or promoter region of a disease-related molecule. An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified cDNA from each segment is determined and compared to a standard curve to determine the amount of the target nucleic acid segment that was present in the sample prior to amplification. In some embodiments, the expression of a “housekeeping” gene or “internal control” can also be evaluated. These terms include any constitutively or globally expressed gene whose presence enables an assessment of mRNA levels provided herein. Such an assessment includes a determination of the overall constitutive level of gene transcription and a control for variations in RNA recovery. Exemplary housekeeping genes include tubulin, glyceraldehyde-3-phosphate-dehydrogenase (GAPDH), beta-actin, and 18S ribosomal RNA.

Integrating Molecular Data with Patient Outcomes Data, Electronic Health Records and/or Health Insurance Claims Data

In various examples, the implementations described herein can integrate molecular data from a variety of sample types, the data can comprise genetic, epigenetic, transcriptomic, epitranscriptomic, proteomic, fragmentomic, and/or chromatin interactions, transcription factor binding sites (TFBS), fragmentomic patterns, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3 Ac and H4Ac, H3K4me1, H3K4me2, H3K4me3, H2BK120ub, H3.3, and/or H3S10ph with patient outcomes data, electronic health records and/or health insurance claims data to: (1) identify common methylation changes associated with specific disease cancer types or subtypes and stages. The goal of this analysis is to reveal potential methylation biomarkers for disease treatment selection. (2) Correlate methylation changes with clinical outcomes in disease patients. The goal of this analysis is to understand the impact of these methylation changes on malignant cells, cancer progression, resistance, and treatment response. (3) Integrate the identified methylation changes with existing biological pathways and networks relevant in the disease state. (4) Compare methylation data across different cancer types or subtypes to identify unique and shared mechanisms. The goal of this analysis is to understand cancer heterogeneity and similarities across different cancers. (5) Develop a predictive model using methylation data to predict treatment outcomes, recurrence, or drug resistance. The goal of this analysis is to help guide personalized treatment strategies. Such analysis can be achieved using a variety of statistical and machine learning models including Linear Regression and Logistic Regression: For continuous and binary outcomes, respectively, to model the relationship between methylation levels at specific sites and the presence or severity of cancer. Cox Proportional Hazards Model, can be useful for survival analysis to correlate methylation levels with the time to event data, such as time to cancer recurrence or progression. Mixed Models are helpful when the data comprises multiple measurements or hierarchical structures, mixed models can account for the correlation within subjects or groups. Multivariate Additionally, analysis like principal component analysis (PCA) or partial least squares regression (PLSR) can reduce dimensionality and identify patterns in methylation data that correlate with cancer types.

Other methods employed to explore relationships or correlations between methylation data and cancer type/subtype include machine learning models. For example, to handle complex, non-linear relationships we employ Decision Trees and Random Forests these are particularly helpful in situations where the association between the methylation status of certain genes (or CpG sites) and cancer characteristics does not follow a straight-line pattern. For example, to model interaction effects i.e., methylation at one site might affect the impact of methylation at another site. Alone or in one or more combinations these models can identify specific methylation sites that are important for classifying cancer types. In some embodiments, Support Vector Machines (SVM) can be employed for classification tasks, including distinguishing between different types of cancer based on methylation patterns. In yet other embodiments, for example when the data is sufficiently large, deep learning approaches (e.g., convolutional neural networks for structured data like methylation arrays) can capture complex patterns including interactions in the data. Methods such as Gradient Boosting Machines (GBM) including models like XGBoost, LightGBM, and CatBoost can provide robust predictive models for cancer classification based on methylation data. These models include sequential addition of weak learners (e.g., decision trees) in such a way that each new tree corrects the errors made by the previous ones. GBMs can handle various types of data, including categorical and continuous variables. In additional embodiments, Cluster Analysis are employed to identify subgroups within cancer types that share similar methylation patterns. These include unsupervised learning models, including K-means clustering or hierarchical clustering.

In some embodiments, the cell-free DNA is from a subject having or suspected of having cancer and/or the cell-free DNA includes DNA from cancer cells. In some embodiments, the DNA is partitioned into a first subsample and a second subsample, wherein the first subsample comprises DNA with a nucleotide modification (e.g., a cytosine modification) in a greater proportion than the second subsample, and the first subsample is subjected to a procedure that affects a first nucleobase in the DNA differently from a second nucleobase in the DNA of the first subsample, and the DNA is sequenced in a manner that distinguishes the first nucleobase from the second nucleobase in the DNA of the first subsample.

### Epigenetic Therapeutic Drugs

The intricate interplay of chemical modifications to DNA and histone proteins, known as the epigenome, plays a significant role in modulating gene expression in health and disease including MRD and metastasis. Epigenetic changes in conjunction with genetic alterations contribute to the acquisition of cancer hallmarks such as sustaining proliferative signaling, evading growth suppressors, resisting cell death, enabling replicative immortality, inducing angiogenesis, and activating invasion and metastasis (Hanahan, D. 2022). Given the reversible nature of epigenetic modifications, understanding these mechanisms offers promising avenues for therapeutic intervention, with several epigenetic drugs already approved or in clinical trials for the treatment of cancer. In particular, drugs targeting epigenetic enzymes such as DNA methyltransferase, histone deacetylase, isocitrate dehydrogenase, and enhancer of zeste homolog 2 may offer highly tailored therapeutic options do specifically target residual malignant cells in subjects with MRD.

### FDA Approved Epigenetic Drugs

Epigenetic biomarkers for targeted therapy may be particularly valuable for MRD patients who do not exhibit a genetic druggable target. The FDA has approved several epigenetic drugs including DNA methyltransferase (DNMT) inhibitors, Histone deacetylase (HDAC) inhibitors, Lysine methyltransferase inhibitors, Lysine demethylase inhibitors, and Bromodomain inhibitors.

5-azacytidine is a DNA methyltransferase inhibitor used for myelodysplastic syndrome. Decitabine is also a DNA methyltransferase inhibitor used for myelodysplastic syndrome. Vorinostat, an HDAC inhibitor, is indicated for cutaneous T-cell lymphoma, while romidepsin, another HDAC inhibitor, is also used for cutaneous T-cell lymphoma. Belinostat, an HDAC inhibitor, is prescribed for peripheral T-cell lymphoma, and panobinostat, also an HDAC inhibitor, is used for multiple myeloma. Tazemetostat, an EZH2 (KMT inhibitor), is indicated for epithelioid sarcoma and follicular lymphoma. Ivosidenib, an IDH inhibitor, is used for acute myeloid leukemia and cholangiocarcinoma, while enasidenib, another IDH inhibitor, is used for acute myeloid leukemia. Tranylcypromine, a lysine demethylase inhibitor, is indicated for acute myeloid leukemia and myelodysplastic syndrome. Chidamide, an HDAC inhibitor, is used for breast cancer and peripheral T-cell lymphoma, while valemetostat, an EZH2/EZH1 inhibitor, is prescribed for adult T-cell leukemia/lymphoma. Azacitidine, a DNMT1 inhibitor, is used for juvenile myelomonocytic leukemia. Decitabine, another DNMT1 inhibitor, is indicated for myelodysplastic syndrome and chronic myelomonocytic leukemia. Additionally, 5-azacytidine, as a DNA methyltransferase inhibitor, is used for colorectal cancer (immunotherapy sensitization). Decitabine, also a DNA methyltransferase inhibitor, enhances PD-L1 efficacy and immune response in colorectal cancer. Tazemetostat, as an EZH2 inhibitor, has potential to enhance immunotherapy in colorectal cancer. Lastly, TMP195, an HDAC inhibitor, is used for colorectal cancer, focusing on macrophage polarization and enhancing PD-1 inhibitors.

DNA Methyltransferase Inhibitors (DNMTi)-inhibit DNA methyltransferases, enzymes that add methyl groups to DNA, typically silencing gene expression. DNMTi drugs include Azacitidine (Vidaza) which was approved for the treatment of myelodysplastic syndromes (MDS) and Decitabine (Dacogen) also approved for MDS. Histone Deacetylase Inhibitors (HDACi) inhibit histone deacetylases, enzymes that remove acetyl groups from histone proteins, typically leading to a closed chromatin structure and gene silencing. Inhibiting these enzymes can reactivate silenced genes beneficial in cancer treatment. HDACi drugs include Vorinostat (Zolinza) approved for the treatment of cutaneous T cell lymphoma (CTCL), Romidepsin (Istodax) approved for CTCL and peripheral T-cell lymphoma (PTCL), Belinostat (Beleodaq) approved for PTCL, and Panobinostat (Farydak) approved for multiple myeloma in combination with bortezomib and dexamethasone. EZH2 Inhibitors, EZH2 is a component of the polycomb repressive complex 2 (PRC2) that methylates histone H3 on lysine 27 (H3K27me3), leading to gene silencing. EZH2 Inhibitors include Tazemetostat (Tazverik) approved for the treatment of epithelioid sarcoma and follicular lymphoma. Additional epigenetic drugs include Bromodomain Inhibitors that target bromodomains, which recognize acetylated lysine residues on histone tails, influencing chromatin structure and gene expression; however, currently there are no bromodomain inhibitors approved by the FDA.

Partitioning the Sample into a Plurality of Subsamples

In certain exemplary embodiments, described herein, a population of different forms of nucleic acids, or other molecular classes such as proteins and metabolites, (e.g., hypermethylated and hypomethylated DNA in a sample, such as a captured set of cfDNA as described herein) can be physically partitioned based on one or more biochemical features of the molecules prior to further analysis, e.g., differentially modifying or isolating a nucleobase or molecule, tagging, and/or sequencing. Additionally the population of different forms of biochemical changes in nucleic acids and molecular classes may include transcription factor binding sites (TFBS), mRNA expression, RNA modifications, RNA editing and noncoding RNA regulations, protein modifications including histone acetylation, SUMOylation, phosphorylation, and ubiquitination, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3 Ac and H4Ac, H3K4me1, H3K4me2, H3K4me3, H2BK120ub, H3.3, H3S10ph. This approach can be used to determine, for example, whether certain sequences are hypermethylated or hypomethylated or contain a specific histone modification or functional element such as a TFBS. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved. In some embodiments of the disclosure each partition may comprise different forms of nucleic acids or molecular classes comprising different biochemical features, including transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3 Ac and H4Ac, H3K4me1, H3K4me2, H3K4me3, H2BK120ub, H3.3, H3S10ph. This approach can be used to determine, for example, whether certain sequences are hypermethylated or hypomethylated or contain a specific histone modification or functional element such as a TFBS. Additionally, molecular classes such as proteins can be partitioned based on biochemical features such as histone acetylation, SUMOylation, phosphorylation, and ubiquitination modifications.

In some instances, a heterogeneous nucleic acid sample is partitioned into two or more partitions. For instance, a minimum of three partitions, extending to four, five, six, seven, up to any number of partitions, where the total number can be any non-negative real number). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means.

Examples of molecular attributes that can be used for partitioning include sequence length, methylation level, nucleosome binding, sequence mismatch, immunoprecipitation, and/or proteins that bind to DNA. Additional characteristics that can be used for partitioning include, transcription factor binding sites (TFBS), fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph, type of nucleic acid for example RNA, mRNA, cDNA, or DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced.

In some embodiments, a population of different nucleic acids is partitioned into two or more different partitions. Each partition is representative of a different nucleic acid form, and a first partition (also referred to as a subsample) comprises DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants, a variety of epigenetic marks or functional elements for example TFBS, CTCF binding sites can be performed on a partition-by-partition level, as well as whole nucleic acid population level. For example, analysis can include in silico analysis to determine genetic variants, such as CNV, SNV, indel, fusion in nucleic acids in each partition. Additionally, analysis can include in silico analysis to determine transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine.

The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine.

Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides.

Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted.

In some instances, the final partitions are representative of nucleic acids having different extents of modifications (over representative or under representative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. Similarly, histone marks, or TFBS can be can be separated using appropriate antibodies specific to the biochemical properties of each molecule, for example Anti-CTCF, Anti-Pol II (RNA Polymerase II), Anti-H3K4me3 (Histone H3 tri-methylated at lysine 4), Anti-H3K27me3 (Histone H3 tri-methylated at lysine 27), Anti-H3K9me3 (Histone H3 tri-methylated at lysine 9), Anti-H3K27ac (Histone H3 acetylated at lysine 27), Anti-H3K4me1 (Histone H3 mono-methylated at lysine 4), Anti-H3K36me3 (Histone H3 tri-methylated at lysine 36). The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher level of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In certain exemplary embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

### Adapter Ligation or Addition; Tagging

In certain exemplary embodiments, the disclosed methods comprise adding adapters to molecular classes including DNA or RNA (such as cDNA, cell-free DNA, or fragmented genomic DNA). In some embodiments, adapters are added to the DNA before or after subjecting the DNA to a procedure that affects a first nucleobase in the DNA differently from a second nucleobase in the DNA, such as after the subjecting. When adapters are added to the DNA before subjecting the DNA to a procedure that affects a first nucleobase in the DNA differently from a second nucleobase in the DNA, they may comprise nucleotides that are resistant to the procedure. For example, where the procedure comprises contacting the DNA with a deaminase, the adapters may comprise deaminase-resistant cytosines such as 5-ghmC or 5-propynyl cytosine. Similarly, where the procedure comprises contacting the DNA with bisulfite, the adapters may comprise bisulfite-resistant cytosines such as 5-mC or 5-hmC. In some embodiments, adapters may be added or to DNA concurrently with an amplification procedure, e.g., by providing the adapters in a 5′ portion of a primer (where PCR is used, this can be referred to as library prep-PCR or LP-PCR), before or after an amplification step. In some embodiments, adapters are added by other approaches. In some such methods, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified. In some embodiments, end repair of the DNA is performed prior to addition of adapters.

In some embodiments, the single-stranded DNA library preparation is performed in a one-step combined phosphorylation/ligation reaction, e.g., as described in Troll et al., BMC Genomics, 20:1023 (2019), available at https://doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, following attachment of adapters, the nucleic acids, including DNA or RNA are subject to amplification. The amplification can use, e.g., universal primers that recognize primer binding sites in the adapters.

In some embodiments, the DNA and/or RNA is linked at both ends to Y-shaped adapters including primer binding sites and tags. In some such embodiments, the DNA is amplified.

In embodiments of the disclosed methods, a target nucleic acid comprises a 5′ adapter, a 3′ adapter, or both a 5′ adapter and a 3′ adapter. In such embodiments, the 5′ adapter, or both the 5′ adapter and the 3′ adapter comprise at least one sequence that is recognized by at least one restriction enzyme, such as a restriction enzyme described elsewhere herein. In particular embodiments, the 5′ adapter is downstream of an oligonucleotide probe binding site within the target nucleic acid.

Tagging nucleic acid molecules including DNA or RNA molecules is a procedure in which a tag is attached to or associated with the nucleic acid molecules. Such tags can be molecules, such as nucleic acids, containing information that indicates a feature of the molecule with which the tag is associated. Tags can allow one to differentiate molecules from which sequence reads originated. For example, molecules can bear a sample tag (which distinguishes molecules in one sample from those in a different sample) or a molecular tag/molecular barcode/barcode (which distinguishes different molecules from one another in both unique and non-unique tagging scenarios). For methods that involve a partitioning step, a partition tag (which distinguishes molecules in one partition from those in a different partition) may be included. In some embodiments, adapters added to DNA molecules comprise tags. In some such embodiments, the tag comprises one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member. Tags comprising barcodes can be incorporated into or otherwise joined to adapters. Tags can be incorporated by ligation, overlap extension PCR among other methods.

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites. In some embodiments, adapters include the same primer binding site.

In certain embodiments of non-unique tagging, the number of different tags used can be sufficient that there is a very high likelihood (e.g., at least 99%, at least 99.9%, at least 99.99% or at least 99.999% that all molecules of a particular group bear a different tag. In some embodiments comprising barcode attachment, e.g., randomly, to both ends of a molecule, the combination of barcodes, together, constitutes a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by U.S. patent applications Ser. No. 20/010,053519, 20030152490, 20110160078, and U.S. Pat. Nos. 6,582,908 and 7,537,898 and 9,598,731. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample

In some embodiments, 20-50 different tags (e.g., barcodes) are ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

### Tagging of Partitions

In certain exemplary embodiments, two or more partitions, e.g., each partition, is/are differentially tagged. Tags or indexes can be molecules, such as nucleic acids, containing information that indicates a feature of the molecule with which the tag is associated. For example, molecules can bear a sample tag or sample index (which distinguishes molecules in one sample from those in a different sample), a partition tag (which distinguishes molecules in one partition from those in a different partition) and/or a molecular tag/molecular barcode/barcode (which distinguishes different molecules from one another (in both unique and non-unique tagging scenarios). In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member.

Tags can be used to label the individual polynucleotide population partitions so as to correlate the tag (or tags) with a specific partition. Alternatively, tags can be used in embodiments of the invention that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In one embodiment, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics for example transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3 Ac and H4Ac, H3K4me1, H3K4me2, H3K4me3, H2BK120ub, H3.3, H3S10ph, the final tagged molecules in the library may no longer possess that characteristic. In additional examples, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4 . . . n, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads to detect genetic variants can be performed on a partition-by-partition level, as well as a whole nucleic acid population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

### Alternative Methods of Modified Nucleic Acid Analysis

In some embodiments the adapters are added to the nucleic acids after partitioning the nucleic acids, in other embodiments the adapters may be added to the nucleic acids prior to partitioning the nucleic acids based on molecular characteristics for example transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph. In some such methods, a population of nucleic acids bearing the modification to different extents (e.g., 0, 1, 2, 3, 4, or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site. Following attachment of adapters, the nucleic acids are contacted with an agent that preferentially binds to nucleic acids bearing the modification (such as the previously described such agents). The nucleic acids are partitioned into at least two subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following partitioning, the first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The nucleic acids are then amplified from primers binding to the primer binding sites within the adapters. Following amplification, the different partitions can then be subject to further processing steps, which typically include further (e.g., clonal) amplification, and sequence analysis, in parallel but separately. Sequence data from the different partitions can then be compared.

In another embodiment, a partitioning scheme can be performed using the following exemplary procedure. Nucleic acids are linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The partition including original DNA molecules with methylation is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase comprises a cytosine modified at the 5 position, and the second nucleobase comprises unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase comprises a cytosine modified at the 5 position, and the second nucleobase comprises unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

In some embodiments, methods disclosed herein comprise a step of subjecting DNA, or a subsample thereof, to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA before library preparation using the DNA, before a first amplification of the DNA, before dividing the DNA into a plurality of subsamples, or any combination thereof. In certain embodiments, the DNA is subjected to the procedure before or after contacting the DNA with a methylation-sensitive nuclease.

In some embodiments, the procedure that affects a first nucleobase of the DNA differently from a second nucleobase of the DNA is performed prior to the sequencing and/or (a) prior to or after the selectively depleting the target nucleic acid comprising the wild-type sequence, the target nucleic acid comprising the converted nucleotide, or the target nucleic acid that does not comprise the converted nucleotide; (b) prior to the amplifying the selectively digested population of target nucleic acids; (c) prior to or after the partitioning the population of target nucleic acids into a plurality of subsamples; and/or (d) prior to or after a step of enriching for one or more sets of target regions of DNA.

In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, such as where one of the first and second nucleobases comprises mC and the other comprises hmC.

In some embodiments, the procedure that affects a first nucleobase in the DNA differently from a second nucleobase in the DNA comprises bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, facilitates identifying positions containing mC or hmC using the sequence reads obtained from the exemplary sample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336:934-937.

In some embodiments, the procedure that affects a first nucleobase in the DNA differently from a second nucleobase in the DNA comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149:1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected.

Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTet1 or a TET2 comprising a T1372S mutation, can be used to convert mC to caC, and then bisulfite treatment can be used to convert C and caC to U while 5cmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

Alternatively, protection of hmC (e.g., using βGT or 5-hydroxymethylcytosine carbamoyltransferase) can be combined with Tet-assisted conversion with a substituted borane reducing agent, e.g. as described above. In this method (TAPS-β), 5hmC can be protected from conversion, for example through glucosylation using β-glucosyl transferase (βGT), forming 5-glucosylhydroxymethylcytosine (5ghmC), or through carbamoylation using 5-hydroxymethylcytosine carbamoyltransferase, forming 5cmC. This is described in Yu et al., Cell 2012; 149:1368-80. Treatment with a TET protein, such as mTet1 or a TET2 comprising a T1372S mutation, then converts mC to caC but does not convert C, 5ghmC, or 5cmC. 5caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either hmC or unmodified C positions. Meanwhile, positions that are read as T are identified as being T, fC, caC, or mC. Performing TAPSβ conversion, such as on a DNA sample as described herein, thus facilitates distinguishing positions containing unmodified C or hmC on the one hand from positions containing mC using the sequence reads obtained from the sample. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12 (17): e4496.

In some embodiments, the procedure that affects a first nucleobase in the DNA differently from a second nucleobase in the DNA comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuO4) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12 (17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the procedure that affects a first nucleobase in the DNA differently from a second nucleobase in the DNA of the first subsample converts a modified nucleoside. In some embodiments, the conversion procedure which converts a modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA. Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36:1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g.,carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using βGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (GT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149:1368-80, and in Yang et al., Bio-protocol, 2023; 12 (17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO4) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO4).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, TET3, or a TET2 comprising a T1372S mutation. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH3CN), lithium borohydride (LiBH4), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088. In some embodiments, the one or more TET enzymes comprise TETcd. TETcd is described in U.S. Pat. No. 10,260,088. In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V1900I, or V1900P TET mutant. In some embodiments, the one or more TET enzymes comprise a V1900 TET2 mutant, such as a V1900A, V1900C, V1900G, V1900I, or V1900P TET2 mutant. Examples of V1900A, V1900C, V1900G, V1900I, and V1900P TET2 mutants. In some embodiments, the V1900 TET mutant has at least 80%, 85%, 90%, 95%, 96%, 97%, 98%, or 99% sequence identity. Position 1900 of the wild-type TET2 sequence corresponds to position 438. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

In some embodiments, the one or more TET enzymes comprise a TET2 enzyme comprising a T1372S mutation, such as TET2-CS-T1372S and TET2-CD-T1372S. Examples of TET2-CS-T1372S and TET2-CD-T1372S. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 (wild type TET2 catalytic domain) of U.S. Pat. No. 10,961,525. Thus, the sequence of a T1372S TET2 catalytic domain may be obtained by changing the threonine at position 258 of U.S. Pat. No. 10,961,525 to serine. TET2 comprising a T1372S mutation is also described in Liu et al., Nat Chem Biol. 2017 February; 13 (2): 181-187. As demonstrated in Liu et al., TET2 comprising a T1372S mutation can more efficiently oxidize 5mC to produce 5-carboxylcytosine (5caC) than other versions of TET2 such as TET2 lacking a T1372S mutation. In some embodiments, the TET2 enzyme is a human TET2 enzyme comprising a T1372S mutation.

Provided herein is a method comprising contacting DNA contacting DNA with a TET2 enzyme comprising a T1372S mutation to oxidize 5-methylcytosine (5mC) and/or 5-hydroxymethylcytosine (5hmC) present in the DNA to 5-carboxycytosine (5caC), subsequently contacting at least a portion of the DNA with a substituted borane reducing agent, thereby converting 5-caC in the DNA to dihydrouracil (DHU), thereby producing treated DNA, and sequencing at least a portion of the treated DNA.

In some embodiments, the procedure that affects a first nucleobase in the DNA differently from a second nucleobase in the DNA comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form β-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63:711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enriching/Capturing Step; Amplification; Adaptors; Barcodes

In some embodiments, methods disclosed herein comprise a step of capturing one or more sets of target regions of DNA, such as cfDNA. Capture may be performed using any suitable approach known in the art.

In some embodiments, capturing comprises contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally comprising the first nucleobase (e.g., hmC) from DNA not originally comprising the first nucleobase, such as hmC-seal), capturing may be performed on any, any two, or all of the DNA originally comprising the first nucleobase (e.g., hmC), the DNA not originally comprising the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein comprises contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test fsor perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein.

In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Captured Set

In some embodiments, a captured set of DNA (e.g., cfDNA) is provided. With respect to the disclosed methods, the captured set of DNA may be provided, e.g., by performing a capturing step after a partitioning step as described herein. The captured set may comprise DNA corresponding to a sequence-variable target region set, an epigenetic target region set, or a combination thereof. In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The first and second captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, a 90- to 100-fold greater concentration, a 10- to 20-fold greater concentration, a 10- to 40-fold greater concentration, a 10- to 50-fold greater concentration, a 10- to 70-fold greater concentration, or a 10- to 100-fold greater concentration. The degree of difference in concentrations accounts for normalization for the footprint sizes of the target regions, as discussed in the definition section.

### Epigenetic Target Region Set

The epigenetic target region set may comprise one or more types of target regions likely to differentiate DNA from neoplastic cells in MRD patients. In certain embodiments epigenetic target regions can differentiate DNA from tumor or cancer cells and from healthy cells, e.g., non-neoplastic circulating cells. Exemplary types of such regions are discussed in detail herein. The epigenetic target region set may also comprise one or more control regions, e.g., as described herein. In additional embodiments the epigenetic target region set may comprise different forms of nucleic acids may include transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3 Ac and H4Ac, H3K4me1, H3K4me2, H3K4me3, H2BK120ub, H3.3, H3S10ph. In additional embodiments the epigenetic target region set may comprise one or more epigenetic signatures. In yet other embodiments the epigenetic target region set may comprise one or more biomarkers for epigenetic drugs.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Variable Target Regions

In some embodiments, the epigenetic target region set comprises one or more hypermethylation variable target regions. In general, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In an example, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions include one or more genomic regions, where the cfDNA molecules in those regions do not differ in methylation state in cancer subjects relative to cfDNA from healthy subjects, but the presence/increased quantity of hypermethylated cfDNA in those regions is indicative of a particular tissue type (e.g., cancer origin) and is presented as cfDNA with increased apoptosis (e.g. tumor shedding) into circulation.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions based on colorectal cancer (CRC) studies is provided in Table 2. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 2
Exemplary Hypermethylation Target
Regions based on CRC studies.

| | Additional | |
|---|---|---|
| | Gene | |
| Gene Name | Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | MGMT | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, the hypermethylation variable target regions comprise a plurality of loci listed in Table 2, e.g., at least 10%, 20%, 30%, 40%, 50%, 60%, 70%, 80%, 90%, or 100% of the loci listed in Table 2. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 2, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5 (5): 492-504 (2016); Skvortsova et al., Br. J. Cancer. 94 (10): 1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions based on lung cancer studies is provided in Table 3. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 3
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 3 may be combined with any of the embodiments described above concerning target regions identified in Table 2. In some embodiments, the hypermethylation variable target regions comprise a plurality of loci listed in Table 2 or Table 3, e.g., at least 10%, 20%, 30%, 40%, 50%, 60%, 70%, 80%, 90%, or 100% of the loci listed in Table 2 or Table 3.

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In yet additional embodiments the, epigenetic target region set comprises transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph. This approach can be used to determine, for example, whether certain sequences are hypermethylated or hypomethylated or contain a specific histone modification or functional element such as a TFBS.

### Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells. In an example, hypomethylation variable target regions can include regions that do not necessarily differ in methylation state in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions include one or more genomic regions, where the cfDNA molecules in those regions do not differ in methylation state in cancer subjects relative to cfDNA from healthy subjects, but the presence/increased quantity of hypomethylated cfDNA in those regions is indicative of a particular tissue type (e.g., cancer origin) and is presented as cfDNA with increased apoptosis (e.g. tumor shedding) into circulation.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

### CTCF Binding Sites

CTCF is a DNA-binding protein that contributes to chromatin organization and often colocalizes with cohesin. Perturbation of CTCF binding sites has been reported in a variety of different cancers. See, e.g., Katainen et al., Nature Genetics, doi: 10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Details regarding sequencing-based fragment length analysis are provided in Snyder et al., Cell 164:57-68 (2016); WO 2018/009723; and US20170211143A1, each of which are incorporated herein by reference.

Thus, perturbations of CTCF binding result in variation in the fragmentation patterns of cfDNA. As such, CTCF binding sites represent a type of fragmentation variable target regions.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

Accordingly, in some embodiments, the epigenetic target region set includes CTCF binding regions. In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above.

In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

### Transcriptional Start Sites

Transcriptional start sites may also show perturbations in neoplastic cells. For example, nucleosome organization at various transcription start sites in healthy cells of the hematopoietic lineage-which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites that do not necessarily differ epigenetically in cancerous tissue relative to DNA from healthy tissue of the same type, but do differ epigenetically (e.g., with respect to nucleosome organization) relative to cfDNA that is typical in healthy subjects. Where, for example, the presence of a cancer results in increased cell death such as apoptosis of cells of the tissue type corresponding to the cancer, such a cancer can be detected at least in part using such transcription start sites.

Thus, perturbations of transcription start sites also result in variation in the fragmentation patterns of cfDNA. As such, transcription start sites also represent a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34 (Database issue): D86-D89 (2006), which is incorporated herein by reference.

Accordingly, in some embodiments, the epigenetic target region set includes transcriptional start sites. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

### Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

### Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### Sequence-Variable Target Region Set

In some embodiments, the sequence-variable target region set comprises a plurality of regions known to undergo somatic mutations in cancer.

In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 4 and Table 5. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 4. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, or 70 of the SNVs of Table 4. In some embodiments, a sequence-variable target region set used in the methods of the present disclosure comprises at least 1, at least 2, at least 3, at least 4, at least 5, or 6 of the fusions of Table 4. In some embodiments, a sequence-variable target region set used in the methods of the present disclosure comprise at least a portion of at least 1, at least 2, or 3 of the indels of Table 4. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, a sequence-variable target region set used in the methods of the present disclosure comprises at least 1, at least 2, at least 3, at least 4, at least 5, or 6 of the fusions of Table 5. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. Each of these genomic locations of interest may be identified as a backbone region or hot-spot region for a given panel. An example of a listing of hot-spot genomic locations of interest may be found in Table 6. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6. Each hot-spot genomic region is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic region of interest may seek to capture.

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 5

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 6

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total target | | |
| | | | | region) | | |
| | | | | 16330 | | |
| | | | | (total probe | | |
| | | | | coverage) | | |

Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above.

### Subjects

In some embodiments, the DNA (e.g., cfDNA or genomic DNA from tissue) is obtained from a subject having a cancer. In some embodiments, the DNA (e.g., cfDNA or genomic DNA from tissue) is obtained from a subject suspected of having a cancer. In some embodiments, the DNA (e.g., cfDNA or genomic DNA from tissue) is obtained from a subject having a tumor. In some embodiments, the DNA (e.g., cfDNA or genomic DNA from tissue) is obtained from a subject suspected of having a tumor. In some embodiments, the DNA (e.g., cfDNA or genomic DNA from tissue) is obtained from a subject having neoplasia. In some embodiments, the DNA (e.g., cfDNA, or genomic DNA from tissue) is obtained from a subject suspected of having neoplasia. In some embodiments, the DNA (e.g., cfDNA, or genomic DNA from tissue) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

### Sequencing

In general, sample nucleic acids flanked by adapters with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously. Additionally, sequencing chemistries can include Illumina sequencing (MiSeq, HiSeq, NextSeq. NovaSeq, MiniSeq, iSeq 100), Oxford Nanopore sequencing (MinION, GridION, PromethION, Flongle), Sanger sequencing (ABI 3730x1), Ion Torrent sequencing (Ion PGM, Ion S5, Ion GeneStudio S5), PacBio SMRT sequencing (Sequel, Sequel Ile), Illumina NovaSeq 6000, PacBio HiFi sequencing (Sequel II and Sequel Ile Systems using HiFi chemistry), Element Biosciences (AVITI System), Ultima Genomics (Ultima Sequencing), and Singular Genomics (G4 Sequencing System).

The sequencing reactions can be performed on one or more forms of nucleic acids at least one of which is known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can performed on at least 5, 10, 20, 70, 100, 200 or 500 different genes, or at most 5000, 2500, 1000, 500 or 100 different genes.

Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

### Differential Depth of Sequencing

In some embodiments, nucleic acids corresponding to the sequence-variable target region set are sequenced to a greater depth of sequencing than nucleic acids corresponding to the epigenetic target region set. For example, the depth of sequencing for nucleic acids corresponding to the sequence variant target region set may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

### Analysis

In some embodiments, a method described herein comprises identifying the presence or absence of DNA produced by a tumor (or neoplastic cells, or cancer cells).

The present methods can be used to diagnose presence or absence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

An exemplary method for molecular tag identification of MBD-bead partitioned libraries through NGS which includes a step of subjecting the first subsample to a procedure that affects a first nucleobase in the DNA differently from a second nucleobase in the DNA of the first subsample is as follows:
1. Physical partitioning of an extracted DNA sample (e.g., extracted blood plasma DNA from a human sample, which has optionally been subjected to target capture as described herein) using a methyl-binding domain protein-bead purification kit, saving all elutions from process for downstream processing.
2. Parallel application of differential molecular tags and NGS-enabling adapter sequences to each partition. For example, the hypermethylated, residual methylation (‘wash’), and hypomethylated partitions are ligated with NGS-adapters with molecular tags.
3. Subject hypermethylated partition to a procedure that affects a first nucleobase in the DNA differently from a second nucleobase in the DNA, such as any of those described herein.
4. Re-combining all molecular tagged partitions, and subsequent amplification using adapter-specific DNA primer sequences.
5. Capture/hybridization of re-combined and amplified total library, targeting genomic regions of interest (e.g., cancer-specific genetic variants and differentially methylated regions).
6. Re-amplification of the captured DNA library, appending a sample tag. Different samples are pooled, and assayed in multiplex on an NGS instrument.
7. Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

In some embodiments of methods described herein, including but not limited to the method shown above, the molecular tags consist of nucleotides that are not altered by the procedure that affects a first nucleobase in the DNA differently from a second nucleobase in the DNA, such as any of those described herein (e.g., mC along with A, T, and G where the procedure is bisulfite conversion or any other conversion that does not affect mC; hmC along with A, T, and G where the procedure is a conversion that does not affect hmC; etc.). In some embodiments of methods described herein, including but not limited to the method shown above, the molecular tags do not comprise nucleotides that are altered by the procedure that affects a first nucleobase in the DNA differently from a second nucleobase in the DNA, such as any of those described herein (e.g., the tags do not comprise unmodified C where the procedure is bisulfite conversion or any other conversion that affects C; the tags do not comprise mC where the procedure is a conversion that affects mC; the tags do not comprise hmC where the procedure is a conversion that affects hmC; etc.).

In general, the procedure that affects a first nucleobase in the DNA differently from a second nucleobase in the DNA may instead be performed before the step of parallel application of differential molecular tags and NGS-enabling adapter sequences to each partition. For example, this may be done where the procedure that affects a first nucleobase in the DNA differently from a second nucleobase in the DNA is a separation, such as hmC-seal, and in such a case the separated populations may themselves be differentially tagged relative to each other. Such an exemplary method is as follows:
1. Physical partitioning of an extracted DNA sample (e.g., extracted blood plasma DNA from a human sample, which has optionally been subjected to target capture as described herein) using a methyl-binding domain protein-bead purification kit, saving all elutions from process for downstream processing.
2. Subject hypermethylated partition to a procedure that affects a first nucleobase in the DNA differently from a second nucleobase in the DNA, such as any of those described herein.
3. Parallel application of differential molecular tags and NGS-enabling adapter sequences to each partition. For example, the hypermethylated partition (or where applicable, two or more sub-partitions of the hypermethylated partition), residual methylation (‘wash’) partition, and hypomethylated partition are ligated with NGS-adapters with molecular tags.
4. Re-combining all molecular tagged partitions, and subsequent amplification using adapter-specific DNA primer sequences.
5. Capture/hybridization of re-combined and amplified total library, targeting genomic regions of interest (e.g., cancer-specific genetic variants and differentially methylated regions).
6. Re-amplification of the captured DNA library, appending a sample tag. Different samples are pooled, and assayed in multiplex on an NGS instrument.
7. Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

### Exemplary Partitioning Workflows

Exemplary workflows for partitioning and library preparation are provided herein. In some embodiments, some or all features of the partitioning and library preparation workflows may be used in combination.

### Partitioning

In some embodiments, sample DNA (e.g., between 5 and 200 ng) is mixed with methyl binding domain (MBD) buffer and magnetic beads conjugated with MBD proteins and incubated overnight. Methylated DNA (hypermethylated DNA) binds the MBD protein on the magnetic beads during this incubation. Non-methylated (hypomethylated DNA) or less methylated DNA (intermediately methylated) is washed away from the beads with buffers containing increasing concentrations of salt. For example, one, two, or more fractions containing non-methylated, hypomethylated, and/or intermediately methylated DNA may be obtained from such washes. Finally, a high salt buffer is used to elute the heavily methylated DNA (hypermethylated DNA) from the MBD protein. In some embodiments, these washes result in three partitions (hypomethylated partition, intermediately methylated fraction and hypermethylated partition) of DNA having increasing levels of methylation.

In some embodiments, the three partitions of DNA are desalted and concentrated in preparation for the enzymatic steps of library preparation.

### Library Preparation

In some embodiments (e.g., after concentrating the DNA in the partitions), the partitioned DNA is made ligatable, e.g., by extending the end overhangs of the DNA molecules are extended, and adding adenosine residues to the 3′ ends of fragments and phosphorylating the 5′ end of each DNA fragment. DNA ligase and adapters are added to ligate each partitioned DNA molecule with an adapter on each end. These adapters contain partition tags (e.g., non-random, non-unique barcodes) that are distinguishable from the partition tags in the adapters used in the other partitions. Either before or after making the portioned DNA ligatable and performing the ligation, the hypermethylated partition is subjected to a procedure that affects a first nucleobase in the DNA differently from a second nucleobase in the DNA, such as any of those described herein. Where the procedure that affects a first nucleobase in the DNA differently from a second nucleobase in the DNA further partitions the hypermethylated partition, the ligation of adapters should be performed after the procedure so that the sub-partitions of the hypermethylated partition can be differentially tagged. Then, the three (or more) partitions are pooled together and are amplified (e.g., by PCR, such as with primers specific for the adapters).

Following PCR, amplified DNA may be cleaned and concentrated prior to enrichment. The amplified DNA is contacted with a collection of probes described herein (which may be, e.g., biotinylated RNA probes) that target specific regions of interest. The mixture is incubated, e.g., overnight, e.g., in a salt buffer. The probes are captured (e.g., using streptavidin magnetic beads) and separated from the amplified DNA that was not captured, such as by a series of salt washes, thereby enriching the sample. After the enrichment, the enriched sample is amplified by PCR. In some embodiments, the PCR primers contain a sample tag, thereby incorporating the sample tag into the DNA molecules. In some embodiments, DNA from different samples is pooled together and then multiplex sequenced, e.g., using an Illumina NovaSeq sequencer.

### Additional Features of Certain Disclosed Methods

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders. In some embodiments, the sample is a polynucleotides sample obtained from a tumor tissue biopsy.

### Tissue Sample

In additional embodiments a sample can comprise a tissue sample from epithelial tissue (skin, lining of the digestive tract), connective tissue (bones, tendons, fat and other soft padding tissue), muscle tissue (heart, muscles of the limbs), and/or nervous tissue (brain, spinal cord, nerves). Other tissues include liver tissue including hepatocytes (liver cells), liver sinusoidal endothelial cells, heart for example myocardial cells, endocardial cells, brain including neurons, glial cells (astrocytes, microglia), neural stem cells, kidneys including renal tubular cells, glomerular cells, mesangial cells, lungs including alveolar cells, bronchial epithelial cells, pancreas including islets of Langerhans cells, acinar cells, skin including keratinocytes, melanocytes, fibroblasts, and blood including red blood cells, white blood cells (lymphocytes, monocytes, neutrophils), platelets. In some embodiments, the tissue samples may undergo preservation methods after extraction these include flash freezing, formaldehyde fixation (Formalin-fixed), paraffin embedding, cryopreservation, ethanol fixation, methanol fixation, RNAlater stabilization, Bouin's solution fixation, zinc fixation, and/or freezing in optimal cutting temperature (OCT) compound.

The volume of plasma can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 μg, at least 10 μg, at least 100 μg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 μg, 1 ng, 10 ng, 100 ng, 150 ng, or 200 ng of cell-free nucleic acid molecules. The method can comprise obtaining 1 femtogram (fg) to 200 ng cell-free nucleic acid molecules from samples.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA). In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

Double-stranded DNA molecules in a sample and single stranded nucleic acid molecules converted to double stranded DNA molecules can be linked to adapters at either one end or both ends. Typically, double stranded molecules are blunt ended by treatment with a polymerase with a 5′-3′ polymerase and a 3′-5′ exonuclease (or proof reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### Amplification

Sample nucleic acids flanked by adapters can be amplified by PCR and other amplification methods. Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters, which result in amplification of at least 50, 60, 70 or 80% of double stranded nucleic acids. Preferably the present methods increase the amount or number of amplified molecules relative to control methods performed with T-tailed adapters alone by at least 10, 15 or 20%.

### Tags

Tags comprising barcodes can be incorporated into or otherwise joined to adapters. Tags can be incorporated by ligation, overlap extension PCR among other methods.

### Molecular Tagging Strategies

In some embodiments, the nucleic acid molecules (from the sample of polynucleotides) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, for example, U.S. patents application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

### Bait Sets; Capture Moieties

As discussed above, nucleic acids in a sample can be subject to a capture step, in which molecules having target sequences are captured for subsequent analysis. Target capture can involve use of a bait set comprising oligonucleotide baits labeled with a capture moiety, such as biotin or the other examples noted below. The probes can have sequences selected to tile across a panel of regions, such as genes or different forms of nucleic acids including transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph. In some embodiments, a bait set can have higher and lower capture yields for sets of target regions such as those of the sequence-variable target region set and the epigenetic target region set, respectively, as discussed elsewhere herein. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### Collections of Target-Specific Probes

In some embodiments, a collection of target-specific probes is used in methods described herein. In some embodiments, the collection of target-specific probes comprises target-binding probes specific for a sequence-variable target region set and target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39:8740-8751 (2011); Freier et al., Nucleic Acids Res. 25:4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set are a bait set as discussed above, e.g., probes comprising capture moieties and sequences selected to tile across a panel of regions, such as genes.

In some embodiments, the target-specific probes are provided in a single composition. The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein. Examples of epigenetic target regions can include different forms of nucleic acids including transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph.

In some embodiments, the probes for the epigenetic target region probe set have a footprint of at least 100 kb, e.g., at least 200 kb, at least 300 kb, or at least 400 kb. In some embodiments, the probes for the epigenetic target region set have a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb. In some embodiments, the probes for the epigenetic target region probe set have a footprint of less than 5 kb, at least 5 kb, e.g., at least 10, 20, or 50 kb.

### Hypermethylation Variable Target Regions

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 3, e.g., at least 10%, 20%, 30%, 40%, 50%, 60%, 70%, 80%, 90%, or 100% of the loci listed in Table 3. In some embodiments, the probes specific for hypermethylation variable target regions comprise probes specific for a plurality of loci listed in Table 2 or Table 3, e.g., at least 10%, 20%, 30%, 40%, 50%, 60%, 70%, 80%, 90%, or 100% of the loci listed in Table 2 or Table 3. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

In some embodiments, the probes for the epigenetic target region set comprise probes specific for one or more hypomethylation variable target regions. The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

### CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

### Epigenetic Target Regions

In some embodiments, the probes for the epigenetic target region set include different forms of nucleic acids including transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3 Ac and H4Ac, H3K4me1, H3K4me2, H3K4me3, H2BK120ub, H3.3, H3S10ph.

### Transcriptional Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

### Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

### Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 4. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, or 3 of the indels of Table 4. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, probes specific for the sequence-variable target region set comprise probes specific for at least 1, at least 2, at least 3, at least 4, at least 5, or 6 of the fusions of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Compositions Comprising Captured DNA

Provided herein is a combination comprising first and second populations of captured DNA. The first population may comprise or be derived from DNA with a cytosine modification in a greater proportion than the second population. The first population may comprise a form of a first nucleobase originally present in the DNA with altered base pairing specificity and a second nucleobase without altered base pairing specificity, wherein the form of the first nucleobase originally present in the DNA prior to alteration of base pairing specificity is a modified or unmodified nucleobase, the second nucleobase is a modified or unmodified nucleobase different from the first nucleobase, and the form of the first nucleobase originally present in the DNA prior to alteration of base pairing specificity and the second nucleobase have the same base pairing specificity. The second population does not comprise the form of the first nucleobase originally present in the DNA with altered base pairing specificity. In some embodiments, the cytosine modification is cytosine methylation. In some embodiments, the first nucleobase is a modified or unmodified cytosine and the second nucleobase is a modified or unmodified cytosine. The first and second nucleobase may be any of those discussed herein in the Summary or with respect to subjecting the first subsample to a procedure that affects a first nucleobase in the DNA differently from a second nucleobase in the DNA of the first subsample.

In some embodiments, the first population comprises a sequence tag selected from a first set of one or more sequence tags and the second population comprises a sequence tag selected from a second set of one or more sequence tags, and the second set of sequence tags is different from the first set of sequence tags. The sequence tags may comprise barcodes.

In some embodiments, the first population comprises protected hmC, such as glucosylated hmC.

In some embodiments, the first population was subjected to any of the conversion procedures discussed herein, such as bisulfite conversion, Ox-BS conversion, TAB conversion, ACE conversion, TAP conversion, TAPSβ conversion, or CAP conversion. In some embodiments, the first population was subjected to protection of hmC followed by deamination of mC and/or C.

In some embodiments of the combination, the first population comprises or was derived from DNA with a cytosine modification in a greater proportion than the second population and the first population comprises first and second subpopulations, and the first nucleobase is a modified or unmodified nucleobase, the second nucleobase is a modified or unmodified nucleobase different from the first nucleobase, and the first nucleobase and the second nucleobase have the same base pairing specificity. In some embodiments, the second population does not comprise the first nucleobase. In some embodiments, the first nucleobase is a modified or unmodified cytosine, and the second nucleobase is a modified or unmodified cytosine, optionally wherein the modified cytosine is mC or hmC. In some embodiments, the first nucleobase is a modified or unmodified adenine, and the second nucleobase is a modified or unmodified adenine, optionally wherein the modified adenine is mA.

In some embodiments, the first nucleobase (e.g., a modified cytosine) is biotinylated. In some embodiments, the first nucleobase (e.g., a modified cytosine) is a product of a Huisgen cycloaddition to β-6-azide-glucosyl-5-hydroxymethylcytosine that comprises an affinity label (e.g., biotin).

In any of the combinations described herein, the captured DNA may comprise cfDNA.

The captured DNA may have any of the features described herein concerning captured sets, including, e.g., a greater concentration of the DNA corresponding to the sequence-variable target region set (normalized for footprint size as discussed above) than of the DNA corresponding to the epigenetic target region set. In some embodiments, the DNA of the captured set comprises sequence tags, which may be added to the DNA as described herein. In general, the inclusion of sequence tags results in the DNA molecules differing from their naturally occurring, untagged form.

The combination may further comprise a probe set described herein or sequencing primers, each of which may differ from naturally occurring nucleic acid molecules. For example, a probe set described herein may comprise a capture moiety, and sequencing primers may comprise a non-naturally occurring label.

### Computer Systems

All methods of the present disclosure can be implemented using, or with the aid of, computer systems. For example, such methods may comprise: partitioning the sample into a plurality of subsamples, including a first subsample and a second subsample, wherein the first subsample comprises DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample. The computer system can also include Hardware, for example the hardware components of the system include a central processing unit (CPU), random access memory (RAM), storage devices (such as hard disk drives or solid-state drives), and input/output devices (such as keyboards, mice, and display monitors). The specific configuration of the hardware components may vary based on the System model and user requirements. The System can also include software including operating system software that manages the hardware resources and provides a platform for running application software. In addition to the operating system, the System may come with pre-installed application software designed to meet the needs of specific tasks or industries. Users may also install additional applications as required. Additional features in the computer system can include security systems. For example the system can incorporate multiple layers of security measures, including firewalls, antivirus software, and encryption protocols, to protect against unauthorized access and ensure the confidentiality, integrity, and availability of data. The system can also include connectivity systems comprising various connectivity options, including wired and wireless network connections, to enable communication and data exchange with other systems and devices. Compatibility with standard networking protocols ensures the System can integrate seamlessly into existing network environments. The computer system can also include support and Maintenance systems. For example, a comprehensive support and maintenance services are provided to ensure the system operates efficiently and effectively. This includes technical support, software updates, and hardware repair or replacement services. Other features of the computer system can include compliance protocols. For example, the system can be designed to comply with relevant industry standards and regulatory requirements, ensuring reliability and safety in its operation.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### Practical Applications of the Methods

### Cancer and Other Diseases

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive, or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint, or set of data that is a summation of genetic information derived from different cells in a heterogeneous disease. This set of data may comprise copy number variation, epigenetic variation, and mutation analyses alone or in combination.

The present methods can be used to determine biomarkers, particularly epigenetic biomarkers, for targeted MRD therapy. Additionally, the methods may be used to diagnose, prognose, monitor or observe cancers, metastasis or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the test subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the test subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a test subject and/or classifying a test subject as being a candidate for a subsequent cancer treatment.

Methods of Determining a Risk of Cancer Recurrence in a Test Subject and/or Classifying a Test Subject as being a Candidate for a Subsequent Cancer Treatment.

In some embodiments, a method provided herein is a method of determining a risk of cancer recurrence in a test subject. In some embodiments, a method provided herein is a method of classifying a test subject as being a candidate for a subsequent cancer treatment, wherein the treatment comprises an epigenetics-targeted drug.

Any of such methods may comprise collecting a plurality of molecular classes including DNA, RNA, and/or proteins (e.g., originating or derived from a tumor cell) from the test subject previously diagnosed with MRD at one or more preselected timepoints following one or more previous cancer treatments to the test subject. The subject may be any of the subjects described herein. The nucleic acids may be cfDNA or cfRNA. In certain embodiments the DNA may be obtained from a tissue sample.

Any of such methods may comprise capturing a plurality of sets of target regions from DNA from the subject, wherein the plurality of target region sets comprises a sequence-variable target region set and an epigenetic target region set, whereby a captured set of DNA molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured nucleic acid molecules, whereby a set of sequence information is produced. The captured nucleic acid molecules of the sequence-variable target region set may be sequenced to a greater depth of sequencing than the captured nucleic acid molecules of the epigenetic target region set.

Any of such methods may comprise detecting a presence or absence of molecular profiles, or specific biochemical changes in DNA, RNA and/or proteins originating or derived from residual tumor cells, i.e., cell that persist after curative intent treatment, at preselected timepoint using the set of sequence information. The detection of the presence or absence of DNA originating or derived from a tumor cell may be performed according to any of the embodiments thereof described elsewhere herein.

Methods of determining a risk of cancer recurrence in a test subject may comprise determining a cancer recurrence score that is indicative of the presence or absence, or amount, of the DNA originating or derived from the tumor cell for the test subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a test subject as being a candidate for a subsequent cancer treatment may comprise comparing the cancer recurrence score of the test subject with a predetermined cancer recurrence threshold, thereby classifying the test subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the test subject based on the cancer recurrence score; for example, the DFS period may be 1 year, 2 years, 3, years, 4 years, 5 years, or 10 years.

In some embodiments, the set of sequence information comprises sequence-variable target region sequences, and determining the cancer recurrence score may comprise determining at least a first subscore indicative of the amount of SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., cfDNA found in a blood sample from a healthy subject, or DNA found in a tissue sample from a healthy subject where the tissue sample is of the same type of tissue as was obtained from the test subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the epigenetic target regions, e.g., including one or both of hypermethylation variable target regions and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the first and second subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### Therapies and Related Administration

In certain embodiments, the methods disclosed herein relate to identifying and administering targeted therapies to patients given their epigenetic profiles, and/or the status of a nucleic acid variant as being of somatic or germline origin. In some embodiments, essentially any cancer therapy, particularly MRD targeted therapy may be included as part of these methods, additional therapies may be administered including radiation therapy, chemotherapy. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously, or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations, or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### Kits

Also provided are kits comprising the compositions as described herein. The kits can be useful in performing the methods as described herein. In some embodiments, a kit comprises a first reagent for partitioning a sample into a plurality of subsamples as described herein, such as any of the partitioning reagents described elsewhere herein. In some embodiments, a kit comprises a second reagent for subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity (e.g., any of the reagents described elsewhere herein for converting a nucleobase such as cytosine or methylated cytosine to a different nucleobase). The kit may comprise the first and second reagents and additional elements as discussed below and/or elsewhere herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNFIA, HRAS, IDH1, IDH2, JAK2, JAK3, KDR, KIT, MLH1, MPL, NPM1, PDGFRA, PROC, PTPN11, RET,SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RITI, GATA3, MAP2K1, RHEB, ROS1, ARAF, MAP2K2, NFE2L2, RHOA, and NTRKI. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein. In additional or alternative embodiments, the kits may further comprise a plurality of oligonucleotide probes that selectively hybridize all or some molecules comprising transcription factor binding sites (TFBS), mRNA expression, fragmentomic patterns, fragmentomic levels, fragment end point densities, histone acetylation or methylation marks associated with poised enhancers including H3K4me1, H3K27ac, H3K27me3, promoter regions including H3K4me3, H3/H4ac, H3K4me1, H3K27me3, H3K9me3 and/or H3.3, open chromatin including H3Ac and H4Ac, H3K4me1, H3K4me2, H3K4me3, H2BK120ub, H3.3, H3S10ph.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adaptors having distinct molecular barcodes and identical sample barcodes. The library adaptors may not be sequencing adaptors. For example, the library adaptors do not include flow cell sequences or sequences that permit the formation of hairpin loops for sequencing. The different variations and combinations of molecular barcodes and sample barcodes are described throughout, and are applicable to the kit. Further, in some cases, the adaptors are not sequencing adaptors. Additionally, the adaptors provided with the kit can also comprise sequencing adaptors. A sequencing adaptor can comprise a sequence hybridizing to one or more sequencing primers. A sequencing adaptor can further comprise a sequence hybridizing to a solid support, e.g., a flow cell sequence. For example, a sequencing adaptor can be a flow cell adaptor. The sequencing adaptors can be attached to one or both ends of a polynucleotide fragment. In some cases, the kit can comprise at least 8 different library adaptors having distinct molecular barcodes and identical sample barcodes. The library adaptors may not be sequencing adaptors. The kit can further include a sequencing adaptor having a first sequence that selectively hybridizes to the library adaptors and a second sequence that selectively hybridizes to a flow cell sequence. In another example, a sequencing adaptor can be hairpin shaped. For example, the hairpin shaped adaptor can comprise a complementary double stranded portion and a loop portion, where the double stranded portion can be attached {e.g., ligated) to a double-stranded polynucleotide. Hairpin shaped sequencing adaptors can be attached to both ends of a polynucleotide fragment to generate a circular molecule, which can be sequenced multiple times. A sequencing adaptor can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adaptor can comprise 20-30, 20-40, 30-50, 30-60, 40-60, 40-70, 50-60, 50-70, bases from end to end. In a particular example, the sequencing adaptor can comprise 20-30 bases from end to end. In another example, the sequencing adaptor can comprise 50-60 bases from end to end. A sequencing adaptor can comprise one or more barcodes. For example, a sequencing adaptor can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adaptors can be blunt ended and Y-shaped and can be less than or equal to 40 nucleic acid bases in length. Other variations of the can be found throughout and are applicable to the kit.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

What is claimed is:

1. A method to detect cancer recurrence in a subject, comprising:
(a) obtaining a tissue sample from a tumor in the subject to determine a tumor profile;
(b) determining at least one synthetic biomarker for the subject based on the tumor profile;
(c) obtaining a blood sample from the subject post medical treatment for the tumor, wherein prior to obtaining the blood sample, the subject was administered at least one of the synthetic biomarkers determined in (b); and
(d) detecting the synthetic biomarker in the blood sample, wherein a positive result indicates cancer recurrence, thereby enabling cancer recurrence detection in the subject.

2. The method of claim 1, wherein the synthetic biomarker comprises a reporter attached to a molecule via a cleavable linker such that cleavage of the linker by a cancer specific agent in the subject releases the reporter from the molecule into the circulation and other biofluids in the subject.

3. The method of claim 1, wherein the detecting comprises employing an analytical assay.

4. The method of claim 3, wherein the assay is selected from the group consisting of: mass spectrometry, ELISA, PCR, and a colorimetric assay.

5. The method of any one of the preceding claims, wherein the tumor profile comprises a genetic, epigenetic, transcriptomic, proteomic, metabolomic, fragmentomic, and/or histological profile.

6. The method of claim 1, wherein the synthetic biomarker comprises a carrier particle having one or more cleavable peptide linkers, each linker attaching at least one reporter to the carrier particle such that cleavage of the linker by a tumor-associated protease releases the reporter from the carrier particle into the circulation and other biofluids in the subject.

7. The method of claim 6, wherein the carrier particle comprises iron oxide nanoparticles (IONPs), polyethylene glycol (PEG), or iron oxide nanoworms.

8. The method of claim 6, wherein the reporter comprises at least one expression vector, mass-barcoded reporters, DNA barcodes, ligand-encoded reporters, volatile organic compounds (VOCs), or gold nanocluster.

9. The method of claim 8, wherein the expression vector expresses at least one secreted reporter protein.

10. The method of claim 9, wherein the secreted reporter protein comprises an alkaline phosphatase, gaussian luciferase (Glu), a fluorescent protein, and/or engineered enzyme.

11. The method of claim 9, wherein expression of the secreted reporter protein is driven by a cancer inducible promoter.

12. The method of claim 11, wherein the cancer inducible promoter comprises one or more functional elements inducible by known or unknown cancer specific molecules.

13. The method of claim 12, wherein the known or unknown cancer specific molecule comprise a transcription factor.

14. The method of claim 12, wherein the cancer inducible promoter is activated based on dysregulated gene expression patterns characteristic of cancer cells.

15. The method of any one of claim 11, wherein the cancer inducible promoter comprises TERT, cyclooxygenase 2, surviving, or mucin 1 promoter.

16. The method of claim 8, wherein the mass-barcoded reporters are chemically synthesized small molecules incorporating at least one stable isotope to generate distinct signatures.

17. The method of claim 16, wherein the distinct signatures are detectable by mass spectrometry.

18. The method of claim 16, wherein the stable isotope comprises carbon-13, nitrogen-15, oxygen-18, and/or sulfur-34.

19. The method of any one of claim 2, wherein the agent comprises an enzyme.

20. The method of claim 19, wherein the enzyme comprises a protease, oxidases, transferases, or ligases.
